(19) United States
(12) Patent Application Publication
(10) Pub. No.: US 2021/0189506 A1
(43) Pub. Date: Jun. 24, 2021

(54) DETECTION OF MODIFIED LIVE SWINE INFLUENZA VIRUS VACCINES

(71) Applicant: Boehringer Ingelheim Vetmedica GmbH, Ingelheim am Rhein (DE)

(72) Inventors: Paulina MOLAU-BLAZEJEWSKA, Ilsede (DE); Kent Douglas DOOLITTLE, Huxley, CT (US)

(21) Appl. No.: 16/955,702

(22) PCT Filed: Dec. 14, 2018
(86) PCT No.: PCT/EP18/85035

§ 371 Date: Jun. 18, 2020

(30) Foreign Application Priority Data
Dec. 20, 2017 (EP) 17208943.5

(51) Int. Cl. C12Q 1/6888; C12Q 1/70

(52) U.S. Cl. CPC C12Q 1/6888; C12Q 1/701

(57) ABSTRACT

The present invention relates i.a. to diagnostic kits and methods for detecting an animal vaccinated with a modified live Swine Influenza virus specific vaccine and diagnostic kits and methods for differentiating animals vaccinated with a modified live Swine Influenza virus specific vaccine from animals infected with Swine Influenza virus, respectively.

This application contains a sequence listing in accordance with 37 C.F.R. 1.821-1.825. The sequence listing accompanying this application is hereby incorporated by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is “01-3290-US-1 Sequence Listing_ST25 5Mar21.txt”. The text file is 6.68 KB; it was created on 5 Mar. 2021; and it is being submitted electronically via EFS-Web, concurrent with the filing of the specification.

### BACKGROUND OF THE INVENTION

Influenza infection in pigs was first reported in 1918 and the first swine influenza viruses were isolated from pigs in 1930 (Shope, R. E., 1931, J. Exp. Med. 54:373-385). Swine influenza (SI) is an acute respiratory disease of swine caused by type A and C influenza viruses. Its severity depends on many factors, including host age, virus strain, and secondary infections (Easterday, 1980, Philos Trans R Soc LondB Biol Sci 288:433-7). Before 1998, mainly “classical” H1N1 SI viruses (SIV) were isolated from swine in the United States (Kida et al, 1994, J Gen Virol 75:2183-8; Scholtissek, 1994, Eur J Epidemiol 10:455-8; Olsen et al, 2000, Arch Virol. 145:1399-419). In 1998, SIVs of the subtype H3N2 were isolated in multiple states in the United States.

SIV replication is limited to epithelial cells of the upper and lower respiratory tract of pigs, the nasal mucosa, ethmoid, tonsils, trachea, and lungs, and virus excretion and transmission occur exclusively via the respiratory route. Infectious virus can thus be isolated from the tissues mentioned, as well as from tonsils, bronchoalveolar lavage (BAL) fluid, and nasal, tonsillar, or oropharyngeal swabs (Kristien Van Reeth and Wenjun Ma, 2013, Current Topics in Microbiology and Immunology 370: 173-200).

The influenza virions consist of an internal ribonucleoprotein core (a helical nucleocapsid) containing the single-stranded RNA genome, and an outer lipoprotein envelope lined inside by a matrix protein (M1). The segmented genome of influenza A virus consists of eight molecules of linear, negative polarity, single-stranded RNAs which encode eleven polypeptides, including: the RNA-dependent RNA polymerase proteins (PB2, PB1 and PA) and nucleoprotein (NP) which form the nucleocapsid; the matrix membrane proteins (M1, M2); two surface glycoproteins which project from the lipid containing envelope: hemagglutinin (HA) and neuraminidase (NA); the nonstructural protein (NS1), nuclear export protein (NEP); and the proapoptotic factor PB1-F2.

The type A influenza viruses are divided into 17 HA (hemagglutinin) and 10 NA (Neuraminidase) subtypes which can give rise to many possible combinations (designated as H1N1, H1N2, H2N1, H2N2, H5N1, H5N2 and so on) (Tong et al., 2012, Proc. Natl. Acad. Sci. USA., 109: 4269-4274). The hemagglutinin (HA) plays role in attachment of the virus to the surface of infected cells while the neuraminidase (NA) plays role in release of the progeny viruses from the infected cells therefore NA plays role in spread of the virus (Wang et al., 2009, Biochem. Biophys. Res. Commun., 386: 432-436).

Vaccination is an essential tool to manage herd health. The use of compliance markers for determining if an animal has been properly vaccinated is highly desired by producers. WO 2009/058835 A1 describes that it is nearly impossible to differentiate antibodies resulting from vaccination from antibodies formed in response to natural infection. Further, WO 2009/058835 A1 describes e.g. the use of purified xylanase which was added as a compliance marker to a swine influenza vaccine and describes the detection of antibodies specific for xylanase in blood serum.

Modification of the NS1 can be utilized to produce live attenuated SIVs as described by Solórzano et al. 2005 (J Virol 79:7535-7543), Vincent et al 2012 (Journal of Virology 19: 10597 to 10605) WO 2006/083286 A2 and in WO 2016/137929 A1. Attenuated SIVs expressing NS1-truncated proteins of an H3N2 SIV (sw/Texas/4199-2/98, Tx/98) with 73,99, or 126 amino acids (Tx/98 NS1D73, Tx/98 NS1D99, and Tx/98 NS1D126) have been generated using reverse genetics.

WO 2006/083286 A2 describes modified live swine influenza vaccines, but only describes RT (reverse transcriptase)-PCR experiments for confirming truncation of the NS segment. Further, Pica et al 2012 (Journal of Virology 86: 10293-10301) uses the SYBR qPCR technique for assessing the degree to which the vaccine suppressed replication of the infectious wildtype virus in the lungs of mice. However, none of the documents disclose a method for determining proper vaccination of animals or a method that allows the differentiation between animals infected with SIV and animals vaccinated with a modified live Swine Influenza specific vaccine. Further, none of the documents disclose oligonucleotide probes specific for a modified live Swine Influenza specific vaccine.

Thus, there is a need for methods for determining proper vaccination of animals and a need for methods to allow for the differentiation between animals infected with SIV and animals vaccinated with a modified live Swine Influenza specific vaccine.

### DESCRIPTION OF THE INVENTION

Before the aspects of the present invention are described, it must be noted that as used herein and in the appended claims, the singular forms “a”, “an”, and “the” include plural reference unless the context clearly dictates otherwise. Thus, for example, reference to “an antigen” includes a plurality of antigens, reference to the “virus” is a reference to one or more viruses and equivalents thereof known to those skilled in the art, and so forth. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies as reported in the publications which might be used in connection with the invention. Nothing herein is to be construed as an admission that the invention is not entitled to antedate such disclosure by virtue of prior invention.

The present invention solves the problems inherent in the prior art and provides a distinct advance in the state of the art. Generally, the present invention provides a diagnostic kit for the detection of an animal vaccinated with a modified live Swine Influenza virus specific vaccine comprising an oligonucleotide probe specific for the modified live Swine Influenza specific vaccine comprising at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto.

Advantageously, the experimental data provided by the present invention disclose that the oligonucleotide probe of the present invention can detect the Swine Influenza virus specific vaccine in different samples at various dilutions.

The term “diagnostic kit” refers to a kit for the detection or measurement of said modified live Swine Influenza specific vaccine. The term “kit” as used herein refers to a collection of the elsewhere mentioned components in particular the oligonucleotide probe specific for the modified live Swine Influenza virus specific vaccine. The kit may also comprise an oligonucleotide probe specific for the Swine Influenza virus, the primers as described elsewhere herein, buffers, instruction letter and the alike. Said components may or may not be packaged together. The components of the kit may be comprised by separate vials (i.e. as a kit of separate parts) or provided in a single vial. Moreover, it is to be understood that the kit of the present invention is to be used for practicing the methods referred to herein. It is, preferably, envisaged that all components are provided in a ready-to-use manner for practicing the methods referred to herein. Further, the kit preferably contains instructions for carrying out the said methods. The instructions can be provided by a user's manual in paper- or electronic form. For example, the manual may comprise instructions for interpreting the results obtained when carrying out the aforementioned methods using the kit of the present invention.

The term “animal” refers to animals, preferably to mammals such as mice, rats, guinea pigs, rabbits, hamsters, swine, sheep, dogs, cats, horses, monkeys, or cattle and, also preferably, to humans. More preferably, the subject is a swine. Preferably, the swine is a piglet of about 1 week of age and younger, more preferably 3 weeks of age and younger, most preferably 6 weeks of age and younger.

The term “modified live” means that the virus has been reduced in virulence by any of several methods known in the art such, including but not limited to repeated passage in cell culture; forced adaptation to growth at normally-restrictive temperatures; treatment with chemical mutagens to force high numbers of mutations and selection for the desired characteristics; and deletion or insertion of genes using recombinant technology. Preferably, the virus has been reduced in virulence by truncation of the NS-1 protein.

The term “swine influenza virus” is known by the person skilled in the art. The term swine influenza virus refers to a type A or type C influenza virus from the family orthomyxovirus that causes swine influenza. Preferably, the term swine influenza virus refers to a type A virus, a Swine Influenza A virus (SIAV). While orthomyxovirus has three groups: type A, type B and type C, only type A and type C influenza viruses infect pigs. Subtypes of swine influenza virus include H1N1, H1N2, H3N2, and H3N1. H9N2 and H5N1 can also be found in pigs. Preferably, a swine influenza virus is an influenza virus that has been isolated from swine. A swine influenza virus contains a swine NS1 gene. Representative swine NS1 genes can be found in public sequence databases such as Genbank and include, but are not limited to, Genbank Accession No. AJ293939 (A/swine/Italy/13962/95(H3N2)) and Genbank Accession No. AJ344041 (A/swine/Cotes d'Armor/1121/00(H1N1)). Examples of swine influenza virus variants include, but are not limited to, A/Swine/Colorado/1/77, A/Swine/Colorado/23619/99, A/Swine/Cote d'Armor/3633/84, A/Swine/England/195852/92, A/Swine/Finistere/2899/82, A/Swine/Hong Kong/10/98, A/Swine/Hong Kong/9/98, A/Swine/Hong Kong/81/78, A/Swine/Illinois/100084/01, A/Swine/Illinois/100085A/01, A/Swine/Illinois/21587/99, A/Swine/Indiana/1726/88, A/Swine/Indiana/9K035/99, A/Swine/Indiana/P12439/00, A/Swine/Iowa/30, A/Swine/Iowa/15/30, A/Swine/Iowa/533/99, A/Swine/Iowa/569/99, A/Swine/Iowa/3421/90, A/Swine/Iowa/8548-1/98, A/Swine/Iowa/930/01, A/Swine/Iowa/17672/88, A/Swine/Italy/1513-1/98, A/Swine/Italy/1523/98, A/Swine/Korea/CY02/02, A/Swine/Minnesota/55551/00, A/Swine/Minnesota/593/99, A/Swine/Minnesota/9088-2/98, A/Swine/Nebraska/1/92, A/Swine/Nebraska/209/98, A/Swine/Netherlands/12/85, A/Swine/North Carolina/16497/99, A/Swine/North Carolina/35922/98, A/Swine/North Carolina/93523/01, A/Swine/North Carolina/98225/01, A/Swine/Oedenrode/7C/96, A/Swine/Ohio/891/01, A/Swine/Oklahoma/18717/99, A/Swine/Oklahoma/18089/99, A/Swine/Ontario/01911-1/99, A/Swine/Ontario/01911-2/99, A/Swine/Ontario/41848/97, A/Swine/Ontario/97, A/Swine/Quebec/192/81, A/Swine/Quebec/192/91, A/Swine/Quebec/5393/91, A/Swine/Taiwan/7310/70, A/Swine/Tennessee/24/77, A/Swine/Texas/4199-2/98, A/Swine/Wisconsin/125/97, A/Swine/Wisconsin/136/97, A/Swine/Wisconsin/163/97, A/Swine/Wisconsin/164/97, A/Swine/Wisconsin/166/97, A/Swine/Wisconsin/168/97, A/Swine/Wisconsin/235/97, A/Swine/Wisconsin/238/97, A/Swine/Wisconsin/457/985 A/Swine/Wisconsin/458/98, A/Swine/Wisconsin/464/98 and A/Swine/Wisconsin/14094/99.

In one aspect of the present invention said Swine Influenza virus is a Swine Influenza A virus.

The term “vaccine” refers to a composition that comprises at least one antigen, which elicits an immunological response in the host to which the vaccine is administered. Such immunological response may be a cellular and/or antibody-mediated immune response to the immunogenic composition of the invention. Preferably, the vaccine induces an immune response and, more preferably, confers protective immunity against one or more of the clinical signs of a SIV infection. The host may also be described as “subject”. Preferably, any of the hosts or subjects described or mentioned herein is an animal.

Usually, a “vaccine” includes but is not limited to one or more of the following effects: the production or activation of antibodies, B cells, helper T cells, suppressor T cells, and/or cytotoxic T cells and/or gamma-delta T cells, directed specifically to an antigen or antigens included in the vaccine of the invention. Further, the host will display either a protective immunological response or a therapeutically response.

A “protective immunological response” or “protective immunity” will be demonstrated by either a reduction or lack of clinical signs normally displayed by an infected host, a quicker recovery time and/or a lowered duration of infectivity or lowered pathogen titer in the tissues or body fluids or excretions of the infected host.

The term “oligonucleotide probe” refers to a naturally occurring or synthetic polymer of nucleotides capable of interacting with a target nucleic acid.

In general, the nucleotides comprising an oligonucleotide are naturally occurring deoxyribonucleotides, such as adenine, cytosine, guanine or thymine linked to T-deoxyribose, or ribonucleotides such as adenine, cytosine, guanine or uracil linked to ribose. However, an oligonucleotide also can contain nucleotide analogs, including non-naturally occurring synthetic nucleotides or modified naturally occurring nucleotides. Such nucleotide analogs are well known in the art and commercially available, as are polynucleotides containing such nucleotide analogs. Oligonucleotides can be synthesized by a conventional method such as a triethyl phosphate method and a phosphoric diester method using e.g., a DNA synthesizer commonly employed.

“Probes” are molecules capable of interacting with a target nucleic acid, typically in a sequence specific manner such as through hybridization. The hybridization of nucleic acids is well understood in the art. Typically a probe can be made from any combination of nucleotides or nucleotide derivatives or analogs available in the art.

Preferably, an oligonucleotide probe as meant herein has between 15 and 50 nucleotides in length, more preferably between 18 and 40 nucleotides in length, and most preferably between 25 and 35 nucleotides in length. Preferably, the oligonucleotide is a single-stranded oligodesoxyribonucleotide. However, due to self-complementarity the oligonucleotide may be partially double-stranded under certain conditions (depending on, e.g., the sequence of the oligonucleotide, the salt concentration and the temperature).

Preferably, the oligonucleotide probe of the present invention is a single stranded nucleic acid capable of forming a double stranded molecule (hybrid) by hybridizing specifically to a product (amplicon) amplified by use of the corresponding oligonucleotide primer pair. Preferably, the single stranded nucleic acid is a single stranded DNA.

The term “sequence identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

The terms “sequence identity” or “percent identity” are used interchangeably herein. For the purpose of this invention, it is defined here that in order to determine the percent identity of two amino acid sequences or two nucleic acid sequences, the sequences are aligned for optimal comparison purposes (e.g., gaps can be introduced in the sequence of a first amino acid or nucleic acid for optimal alignment with a second amino or nucleic acid sequence). The amino acid or nucleotide residues at corresponding amino acid or nucleotide positions are then compared. When a position in the first sequence is occupied by the same amino acid or nucleotide residue as the corresponding position in the second sequence, then the molecules are identical at that position. The percent identity between the two sequences is a function of the number of identical positions shared by the sequences (i.e., % identity=number of identical positions/total number of positions (i.e. overlapping positions)×100). Preferably, the two sequences are the same length.

A sequence comparison may be carried out over the entire lengths of the two sequences being compared or over fragment of the two sequences. Typically, the comparison will be carried out over the full length of the two sequences being compared. However, sequence identity may be carried out over a region of, for example, twenty, fifty, one hundred or more contiguous amino acid residues.

It is in particular understood in the context of the present invention that the term “identical with the sequence” is equivalent to the term “identical to the sequence”.

As used herein, it is in particular understood that the term “is at least X % identical with the (sequence of) SEQ ID NO:Y” is equivalent to the term “is at least X % identical with the (sequence of) SEQ ID NO:Y over the length of the (sequence of) SEQ ID NO:Y” or to the term “is at least X % identical with the (sequence of) SEQ ID NO:Y over the entire length of the (sequence of) SEQ ID NO:Y”, respectively. In this context, “X” is any number from 90 to 100, in particular any integer selected from 90 to 100, such that “X % identical with the SEQ (sequence)” represents any of the percent sequence identities mentioned herein. Respectively, “Y” in this context is any integer selected from SEQ ID NO:1 to SEQ ID NO:36, such that “SEQ ID NO:Y” represents any of the SEQ ID NOs mentioned herein.

It is furthermore understood that the term “is at least 99% identical”, as described herein, also (in one extreme of the range) comprises and relates to the term “is 100% identical” or “is identical”, respectively.

The skilled person will be aware of the fact that several different computer programs are available to determine the homology between two sequences. For instance, a comparison of sequences and determination of percent identity between two sequences can be accomplished using a mathematical algorithm. In a preferred embodiment, the percent identity between two amino acid or nucleic acid sequences is determined using the Needleman and Wunsch (J. Mol. Biol. (48): 444-453 (1970)) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6. The skilled person will appreciate that all these different parameters will yield slightly different results but that the overall percentage identity of two sequences is not significantly altered when using different algorithms.

The present invention also provides a diagnostic kit for differentiating animals vaccinated with a modified live Swine Influenza virus specific vaccine from animals infected with Swine Influenza virus comprising
a. an oligonucleotide probe specific for the modified live Swine Influenza virus specific vaccine comprising at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto;
b. an oligonucleotide probe specific for the Swine Influenza virus for detecting an infection with Swine Influenza virus.

Advantageously, the experimental data provided by the present invention disclose that the oligonucleotide probe specific for the modified live Swine Influenza virus specific vaccine and the oligonucleotide probe specific for the Swine Influenza virus can be used simultaneously in one experimental set up as there is no evidence of interference between the different probes (WT and MLV) even at high concentrations of virus.

The term “infection” or “infected” refer to the infection of a subject or animal by Swine Influenza virus.

The present invention also provides a method for detecting an animal vaccinated with a modified live Swine Influenza virus specific vaccine in a biological sample comprising the steps of:
a. obtaining a biological sample containing at least one nucleic acid from an animal;
b. providing a forward and a reverse-oligonucleotide primer pair and an oligonucleotide probe specific for the modified live Swine Influenza virus specific vaccine, said oligonucleotide probe comprises at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto;
c. contacting said oligonucleotide primer pair with said biological sample under conditions which allow for amplification of polynucleotides;
d. generating a signal using said oligonucleotide probe specific for the modified live Swine Influenza virus specific vaccine; and
e. detecting said signal, wherein detection of said signal indicates a vaccination with a Swine Influenza virus specific vaccine in the biological sample.

The term “obtaining” may comprise an isolation and/or purification step known to the person skilled in the art, preferably using precipitation, columns etc.

The term “biological sample” refers to a sample of a body fluid, to a sample of separated cells or to a sample from a tissue or an organ. Samples of body fluids can be obtained by well-known techniques and include, preferably, a nasal sample or oral fluid sample (such as nasal swab sample or oral swab sample or tonsillar swab sample or oropharyngeal swab sample or the alike). Tissue or organ samples may be obtained from any tissue or organ by, e.g., biopsy. Preferably, the tissue sample is a respiratory tissue sample or lung sample. Separated cells may be obtained from the body fluids or the tissues or organs by separating techniques such as centrifugation or cell sorting.

The term “nucleic acid” refers to polynucleotides including DNA molecules, RNA molecules, cDNA molecules or derivatives. The term encompasses single as well as double stranded polynucleotides. The nucleic acid of the present invention encompasses isolated polynucleotides (i.e. isolated from its natural context) and genetically modified forms. Moreover, comprised are also chemically modified polynucleotides including naturally occurring modified polynucleotides such as glycosylated or methylated polynucleotides or artificial modified one such as biotinylated polynucleotides. The terms “nucleic acid” and “polynucleotide” also specifically include nucleic acids composed of bases other than the five biologically occurring bases (adenine, guanine, thymine, cytosine and uracil).

The term “cDNA” refers to a complementary DNA which is synthesized from a messenger RNA (mRNA) template in a reaction catalyzed by the enzyme reverse transcriptase. However, the term “cDNA” is well known by the person skilled in the art.

The term “oligonucleotide primer pair” refers to a naturally occurring or synthetic polymer of nucleotides used as a starting molecule for the amplification of a polynucleotide. Preferably, the amplification technique is PCR or qPCR or the alike which is well known to the person skilled in the art and can be used without further ado.

It is to be understood that the oligonucleotide primer may not be 100% complementary to the target sequence, e.g. due to mismatches between the oligonucleotide sequence and the sequence stretch of a target polynucleotide.

Preferably, an oligonucleotide primer as meant herein has between 15 and 35 nucleotides in length, more preferably between 15 and 30 nucleotides in length, and most preferably between 18 and 25 nucleotides in length. Preferably, the oligonucleotide is a single-stranded oligodesoxyribonucleotide. However, due to self-complementarity the oligonucleotide may be partially double-stranded under certain conditions (depending on, e.g., the sequence of the oligonucleotide, the salt concentration and the temperature).

The term “under conditions which allow for amplification of polynucleotides” as used herein is understood by the skilled person. The term relates to a template-dependent process which results in an increase of the amount of a nucleic acid molecule relative to its initial amount. In accordance with the present invention the amplification of a polynucleotide of interest shall allow its detection by any method deemed appropriate and/or, e.g., described herein below. The amplification of a polynucleotide of interest may be carried out by well-known methods, preferably by PCR, but also by reverse transcriptase PCR, real-time PCR, reverse transcriptase real-time PCR, Templex-PCR, nucleic-acid sequence based amplification (NASBA), and isothermal amplification methods using polymerases and specific oligonucleotides as primers. The aforementioned amplification methods are well known in the art. Preferred embodiments of a PCR in the context of the present invention are described in the Examples.

The present invention also provides a method for detecting an animal vaccinated with a modified live Swine Influenza virus specific vaccine in a biological sample comprising the steps of:
a. obtaining a biological sample containing at least one nucleic acid from an animal;
b. providing a forward and a reverse-oligonucleotide primer pair and an oligonucleotide probe specific for the modified live Swine Influenza virus specific vaccine, said oligonucleotide probe comprises at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto;
c. contacting said oligonucleotide primer pair and said oligonucleotide probe with said biological sample under conditions which allow for amplification of polynucleotides;
d. generating an amplification product and an oligonucleotide probe signal; and
e. detecting said oligonucleotide probe signal, wherein detection of the oligonucleotide probe signal indicates a vaccination with a Swine Influenza virus specific vaccine in the biological sample.
However, it has to be understood that also environmental samples can be tested by the method described herein. The present invention also provides a method for detecting a modified live Swine Influenza virus specific vaccine in an environmental sample comprising the steps of:
a. obtaining an environmental sample containing at least one nucleic acid from an animal;
b. providing a forward and a reverse-oligonucleotide primer pair and an oligonucleotide probe specific for the modified live Swine Influenza virus specific vaccine, said oligonucleotide probe comprises at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto;
c. contacting said oligonucleotide primer pair with said environmental sample under conditions which allow for amplification of polynucleotides;
d. generating a signal using said oligonucleotide probe specific for the modified live Swine Influenza virus specific vaccine; and
e. detecting said signal, wherein detection of said signal indicates the presence of a Swine Influenza virus specific vaccine in the environmental sample.

The present invention also provides a method for detecting a modified live Swine Influenza virus specific vaccine in an environmental sample comprising the steps of:
a. obtaining an environmental sample containing at least one nucleic acid from an animal;
b. providing a forward and a reverse-oligonucleotide primer pair and an oligonucleotide probe specific for the modified live Swine Influenza virus specific vaccine, said oligonucleotide probe comprises at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto;
c. contacting said oligonucleotide primer pair and said oligonucleotide probe with said environmental sample under conditions which allow for amplification of polynucleotides;
d. generating an amplification product and an oligonucleotide probe signal; and
e. detecting said oligonucleotide probe signal, wherein detection of said signal indicates the presence of a Swine Influenza virus specific vaccine in the environmental sample.

The present invention also provides a method of differentiating animals vaccinated with a modified live Swine Influenza virus specific vaccine from animals infected with Swine Influenza virus, comprising
a. obtaining a biological sample containing at least one nucleic acid from an animal;
b. providing
   i) at least one forward and one reverse-oligonucleotide primer pair, and,
   ii) an oligonucleotide probe specific for the modified live Swine Influenza virus specific vaccine for detecting a vaccination with a Swine Influenza virus specific vaccine, said oligonucleotide probe comprises at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto, and,
   iii) an oligonucleotide probe specific for the Swine Influenza virus for detecting an infection with Swine Influenza virus;
c. contacting said oligonucleotide primer pair with said biological sample under conditions which allow for amplification of polynucleotides;
d. generating a signal using said oligonucleotide probe specific for the modified live Swine Influenza specific vaccine and/or said oligonucleotide probe specific for the Swine Influenza virus; and
e. detecting said signal, wherein
   i) detection of a signal using said oligonucleotide probe specific for the modified live Swine Influenza specific vaccine indicates a vaccination with a Swine Influenza specific vaccine in the biological sample, and,
   ii) detection of signal using said oligonucleotide probe specific for the Swine Influenza virus indicates an infection with a Swine Influenza virus in the biological sample.

Said method allows discrimination between animals naturally infected with the field virus (disease-associated) and vaccinated animals. A major advantage of this method of differentiating is that it allows the detection of animals (preferably pigs) acutely infected or infected some time (at least ca. 3 weeks) before taking samples in a vaccinated animal population, and thus offers the possibility to monitor the spread or re-introduction of the swine influenza virus in an animal population. Thus, it makes it possible to declare, with a certain level of confidence, that a vaccinated pig population is free of Swine Influenza virus on the basis of laboratory test results.

Differentiating an animal that is infected with field of Swine Influenza virus or vaccinated with a modified live vaccine or detecting an animal vaccinated with a modified live Swine Influenza virus specific vaccine as described herein preferably is provided by RNA isolation of respiratory cells and reverse transcriptase followed by amplification of the cDNA. Using specific primers for the NS segment and the oligonucleotide probe as described herein a PCR or qPCR can be performed.

The present invention also provides a method of differentiating animals vaccinated with a modified live Swine Influenza virus specific vaccine from animals infected with Swine Influenza virus, comprising
a. obtaining a biological sample containing at least one nucleic acid from an animal;
b. providing
   i) at least one forward and one reverse-oligonucleotide primer pair, and,
   ii) an oligonucleotide probe specific for the modified live Swine Influenza virus specific vaccine for detecting a vaccination with a Swine Influenza virus specific vaccine, said oligonucleotide probe comprises at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto, and,
   iii) an oligonucleotide probe specific for the Swine Influenza virus for detecting an infection with Swine Influenza virus;
c. contacting said oligonucleotide primer pair and said oligonucleotide probes with said biological sample under conditions which allow for amplification of polynucleotides;
d. generating an amplification product and an oligonucleotide probe signal; and
e. detecting said oligonucleotide probe signal, wherein
   i) detection of an oligonucleotide probe signal from the oligonucleotide probe specific for the modified live Swine Influenza virus specific vaccine indicates a vaccination with a Swine Influenza specific vaccine in the biological sample, and,
   ii) detection of an oligonucleotide probe signal from the oligonucleotide probe specific for the Swine Influenza virus indicates an infection with a Swine Influenza virus in the biological sample.

The present invention also provides a method for detecting animals vaccinated with a modified live Swine Influenza virus specific vaccine within a group of animals comprising the steps of:
a. obtaining an environmental Sample containing at least one nucleic acid from an animal;
b. providing a forward and a reverse-oligonucleotide primer pair and an oligonucleotide probe specific for the modified live Swine Influenza virus specific vaccine, said oligonucleotide probe comprises at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto;
c. contacting said oligonucleotide primer pair and said oligonucleotide probe with said environmental Sample under conditions which allow for amplification of polynucleotides;
d. generating a signal using said oligonucleotide probe specific for the modified live Swine Influenza specific vaccine; and
e. detecting the oligonucleotide probe signal, wherein the presence of the oligonucleotide probe signal indicates a vaccination with the Swine Influenza virus specific vaccine within said group of animals.

The present invention also provides a method for detecting animals vaccinated with a modified live Swine Influenza virus specific vaccine within a group of animals comprising the steps of:
a. obtaining an environmental Sample containing at least one nucleic acid from an animal;
b. providing a forward and a reverse-oligonucleotide primer pair and an oligonucleotide probe specific for the modified live Swine Influenza virus specific vaccine, said oligonucleotide probe comprises at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto;
c. contacting said oligonucleotide primer pair and said oligonucleotide probe with said environmental Sample under conditions which allow for amplification of polynucleotides;
d. generating an amplification product and an oligonucleotide probe signal; and
e. detecting the oligonucleotide probe signal, wherein the presence of the oligonucleotide probe signal indicates a vaccination with the Swine Influenza virus specific vaccine within said group of animals.

The term “environmental Sample” refers to a sample which has not been taken directly from an animal, but from the environment where the animals are housed. Preferably, the environmental Sample is an air filter sample, a sample of a rope for collecting oral fluid, a sample of a mop pad or sponge. However, the environmental Sample may be any other sample from the environment where the animals are housed such as swabs from the floor, the walls, gates, panels, clothing from staff or feeding/drinking system. Animals infected with Swine Influenza virus or vaccinated with the modified live vaccine are shedding for a few days the wildtype virus and modified live vaccine virus, respectively. Thus environmental Samples can be taken for assessing whether the modified live vaccine virus is present in the environment. A positive test result (presence of the modified live vaccine virus in the environment) implies that the animals housed in the environment have been successfully vaccinated (at least partially). Using the oligonucleotide probe specific for the wildtype virus and having a positive test result (presence of the wildtype virus in the environment) implies that the animals housed in the environment are infected by the wildtype virus.

The present invention also provides a method for determining a ratio between animals vaccinated with a modified live Swine Influenza virus specific vaccine and animals infected with Swine Influenza virus within a group of animals comprising the steps of:
a. obtaining an environmental Sample containing at least one nucleic acid from an animal;
b. providing
   i) at least one forward and one reverse-oligonucleotide primer pair, and,
   ii) an oligonucleotide probe specific for the modified live Swine Influenza virus specific vaccine for detecting a vaccination with a Swine Influenza virus specific vaccine, said oligonucleotide probe comprises at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto, and,
   iii) an oligonucleotide probe specific for the Swine Influenza virus for detecting an infection with Swine Influenza virus;
c. contacting said oligonucleotide primer pair and said oligonucleotide probes with said environmental Sample under conditions which allow for amplification of polynucleotides;
d. generating a signal using said oligonucleotide probe specific for the modified live Swine Influenza specific vaccine and/or said oligonucleotide probe specific for the Swine Influenza virus; and
e. detecting the oligonucleotide probe signal from
   i) the oligonucleotide probe specific for the modified live Swine Influenza virus specific vaccine, and,
   ii) the oligonucleotide probe specific for the Swine Influenza virus;
f. generating a ratio of i) and ii) or ii) and i) of step e.

The present invention also provides a method for determining a ratio between animals vaccinated with a modified live Swine Influenza virus specific vaccine and animals infected with Swine Influenza virus within a group of animals comprising the steps of:
a. obtaining an environmental Sample containing at least one nucleic acid from an animal;
b. providing
   i) at least one forward and one reverse-oligonucleotide primer pair, and,
   ii) an oligonucleotide probe specific for the modified live Swine Influenza virus specific vaccine for detecting a vaccination with a Swine Influenza virus specific vaccine, said oligonucleotide probe comprises at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto, and,
   iii) an oligonucleotide probe specific for the Swine Influenza virus for detecting an infection with Swine Influenza virus;
c. contacting said oligonucleotide primer pair and said oligonucleotide probes with said environmental Sample under conditions which allow for amplification of polynucleotides;
d. generating an amplification product and an oligonucleotide probe signal; and
e. detecting the oligonucleotide probe signal from
   i) the oligonucleotide probe specific for the modified live Swine Influenza virus specific vaccine, and,
   ii) the oligonucleotide probe specific for the Swine Influenza virus;
f. generating a ratio of i) and ii) or ii) and i) of step e.

In case the ratio of the signal of i) and ii) of step e is high, this reflects a vaccination of the animals with the modified live Swine Influenza virus specific vaccine, whereas the infection rate with the wildtype SIV is low. In case the ratio of the signal of i) and ii) of step e is low, this reflects no or low vaccination of the animals with the modified live Swine Influenza virus specific vaccine, whereas the infection rate with the wildtype SIV is high. However, in case the ratio of the signal of i) and ii) of step e is similar, this reflects both vaccinated animals with the modified live Swine Influenza virus specific vaccine and an infection with the wildtype SIV at similar levels.

However, in case the ratio of the signal of ii) and i) of step e is high, this reflects a low vaccination of the animals with the modified live Swine Influenza virus specific vaccine, whereas the infection rate with the wildtype SIV is high. In case the ratio of the signal of ii) and i) of step e is low, this reflects a high vaccination of the animals with the modified live Swine Influenza virus specific vaccine, whereas the infection rate with the wildtype SIV is low. Further, in case the ratio of the signal of ii) and i) of step e is similar, this reflects both vaccinated animals with the modified live Swine Influenza virus specific vaccine and an infection with the wildtype SIV at similar levels.

In one aspect of the present invention step a or c comprises extracting said nucleic acid from said biological sample or said environmental Sample.

The term “extracting” is known to the person skilled in the art and may comprise solubilization, isolation and/or purification steps.

In one aspect of the present invention step a or c comprises a reverse transcription of the RNA.

The term “reverse transcription” is known to the person skilled in the art. The reverse transcription is catalyzed by the enzyme reverse transcriptase. By this reverse transcription from a RNA template cDNA is synthesized.

In one aspect of the present invention said diagnostic kit comprises at least one forward and reverse-oligonucleotide primer pair.

### Oligonucleotide Probe Specific for the Modified Live Swine Influenza Virus Specific Vaccine

In one aspect of the present invention the oligonucleotide probe specific for the modified live Swine Influenza specific vaccine comprising at least twelve, fourteen sixteen or seventeen contiguous nucleotides of the sequence shown in SEQ ID NO:3 (tagatcttgattaattaa) or its reverse complementary sequence (SEQ ID NO:4 ttaattaatcaagatcta) or a sequence having at least 70%, at least 75%, at least 80%, at least 85%, at least 90%, at least 95%, at least 97.5% sequence identity thereto.

In one aspect of the present invention the oligonucleotide probe specific for the modified live Swine Influenza virus specific vaccine comprises at least fourteen contiguous nucleotides of the sequence shown in SEQ ID NO:3 or its reverse complementary sequence (SEQ ID NO:4) or a sequence having at least 70% sequence identity thereto.

In one aspect of the present invention the oligonucleotide probe specific for the modified live Swine Influenza virus specific vaccine comprises at least fifteen contiguous nucleotides of the sequence shown in SEQ ID NO:3 or its reverse complementary sequence (SEQ ID NO:4) or a sequence having at least 70% sequence identity thereto.

In one aspect of the present invention the oligonucleotide probe specific for the modified live Swine Influenza virus specific vaccine comprises at least sixteen contiguous nucleotides of the sequence shown in SEQ ID NO:3 or its reverse complementary sequence (SEQ ID NO:4) or a sequence having at least 70% sequence identity thereto.

In one aspect of the present invention the oligonucleotide probe specific for the modified live Swine Influenza virus specific vaccine comprises at least seventeen contiguous nucleotides of the sequence shown in SEQ ID NO:3 or its reverse complementary sequence (SEQ ID NO:4) or a sequence having at least 70% sequence identity thereto.

In one aspect of the present invention the oligonucleotide probe specific for the modified live Swine Influenza virus specific vaccine comprises a sequence shown in SEQ ID NO:3 or its reverse complementary sequence (SEQ ID NO:4) or a sequence having at least 70% sequence identity thereto.

In one aspect of the present invention the oligonucleotide probe specific for the modified live Swine Influenza virus specific vaccine comprises at least twelve contiguous nucleotides of the sequence shown in SEQ ID NO:5 (agtagatcttgattaattaagagggagc) or SEQ ID NO 7: (atggaaaagtagatcttgattaattaagagg) SEQ ID NO 9: (agtagatcttgattaattaagagggagcaatcg) or SEQ ID NO: 39 (AGTAGATCTTGATTAATTAAGAGGGAGCAATCG) or its complementary reverse sequences (SEQ ID NO:6; SEQ ID NO:8; SEQ ID NO:10; SEQ ID NO:40) or a sequence having at least 70% sequence identity thereto.

In one aspect of the present invention the oligonucleotide probe specific for the modified live Swine Influenza specific vaccine comprising at least twelve, fourteen, sixteen, eighteen, twenty, twenty-two, twenty-four or twenty-six contiguous nucleotides of the sequence shown in SEQ ID NO:5, SEQ ID NO:7, SEQ ID NO:9 or SEQ ID NO: 39 or its complementary reverse sequences (SEQ ID NO:6; SEQ ID NO:8; SEQ ID NO:10; SEQ ID NO: 40) or a sequence having at least 70%, at least 75%, at least 80%, at least 85%, at least 90%, at least 95%, at least 97.5% sequence identity thereto.

In one aspect of the present invention the oligonucleotide probe specific for the modified live Swine Influenza virus specific vaccine comprises at least fourteen contiguous nucleotides of the sequence shown in SEQ ID NO:5, SEQ ID NO:7, SEQ ID NO:9 or SEQ ID NO: 39 or its complementary reverse sequences (SEQ ID NO:6; SEQ ID NO:8; SEQ ID NO:10; SEQ ID NO: 40) or a sequence having at least 70% sequence identity thereto.

In one aspect of the present invention the oligonucleotide probe specific for the modified live Swine Influenza virus specific vaccine comprises at least sixteen contiguous nucleotides of the sequence shown in SEQ ID NO:5, SEQ ID NO:7, SEQ ID NO:9 or SEQ ID NO: 39 or its complementary reverse sequences (SEQ ID NO:6; SEQ ID NO:8; SEQ ID NO:10; SEQ ID NO: 40) or a sequence having at least 70% sequence identity thereto.

In one aspect of the present invention the oligonucleotide probe specific for the modified live Swine Influenza virus specific vaccine comprises at least eighteen contiguous nucleotides of the sequence shown in SEQ ID NO:5, SEQ ID NO:7, SEQ ID NO:9 or SEQ ID NO: 39 or its complementary reverse sequences (SEQ ID NO:6; SEQ ID NO:8; SEQ ID NO:10; SEQ ID NO: 40) or a sequence having at least 70% sequence identity thereto.

In one aspect of the present invention the oligonucleotide probe specific for the modified live Swine Influenza virus specific vaccine comprises at least twenty contiguous nucleotides of the sequence shown in SEQ ID NO:5, SEQ ID NO:7, SEQ ID NO:9 or SEQ ID NO: 39 or its complementary reverse sequences (SEQ ID NO:6; SEQ ID NO:8; SEQ ID NO:10; SEQ ID NO: 40) or a sequence having at least 70% sequence identity thereto.

In one aspect of the present invention the oligonucleotide probe specific for the modified live Swine Influenza virus specific vaccine comprises at least twenty-two contiguous nucleotides of the sequence shown in SEQ ID NO:5, SEQ ID NO:7, SEQ ID NO:9 or SEQ ID NO: 39 or its complementary reverse sequences (SEQ ID NO:6; SEQ ID NO:8; SEQ ID NO:10; SEQ ID NO: 40) or a sequence having at least 70% sequence identity thereto.

In one aspect of the present invention the oligonucleotide probe specific for the modified live Swine Influenza virus specific vaccine comprises at least twenty-four contiguous nucleotides of the sequence shown in SEQ ID NO:5, SEQ ID NO:7, SEQ ID NO:9 or SEQ ID NO: 39 or its complementary reverse sequences (SEQ ID NO:6; SEQ ID NO:8; SEQ ID NO:10; SEQ ID NO: 40) or a sequence having at least 70% sequence identity thereto.

In one aspect of the present invention the oligonucleotide probe specific for the modified live Swine Influenza virus specific vaccine comprises at least twenty-six contiguous nucleotides of the sequence shown in SEQ ID NO:5, SEQ ID NO:7, SEQ ID NO:9 or SEQ ID NO: 39 or its complementary reverse sequences (SEQ ID NO:6; SEQ ID NO:8; SEQ ID NO:10; SEQ ID NO: 40) or a sequence having at least 70% sequence identity thereto.

In one aspect of the present invention the oligonucleotide probe specific for the modified live Swine Influenza virus specific vaccine comprises a sequence shown in SEQ ID NO:5, SEQ ID NO:7, SEQ ID NO:9 or SEQ ID NO: 39 or its complementary reverse sequences (SEQ ID NO:6; SEQ ID NO:8; SEQ ID NO:10; SEQ ID NO: 40) or a sequence having at least 70% sequence identity thereto.

In one aspect of the present invention said sequence identity of the oligonucleotide probe is at least 80%.

In one aspect of the present invention said sequence identity of the oligonucleotide probe is at least 90%.

In one aspect of the present invention said sequence identity of the oligonucleotide probe is at least 95%.

In one aspect of the present invention said sequence identity of the oligonucleotide probe is at least 97.5%.

In one aspect of the present invention the sequence of the oligonucleotide probe specific for the modified live Swine Influenza virus specific vaccine comprises the sequence shown in SEQ ID NO:5 or its complementary reverse sequence (SEQ ID NO:6).

In one aspect of the present invention the sequence of the oligonucleotide probe specific for the modified live Swine Influenza virus specific vaccine comprises the sequence shown in SEQ ID NO:7 or its complementary reverse sequence (SEQ ID NO:8).

In one aspect of the present invention the sequence of the oligonucleotide probe specific for the modified live Swine Influenza virus specific vaccine comprises the sequence shown in SEQ ID NO:9 or its complementary reverse sequence (SEQ ID NO:10).

In one aspect of the present invention the sequence of the oligonucleotide probe specific for the modified live Swine Influenza virus specific vaccine comprises the sequence shown in SEQ ID NO:39 or its complementary reverse sequence (SEQ ID NO:40).

In one aspect of the present invention the oligonucleotide probe specific for the modified live Swine Influenza virus specific vaccine binds to a non-naturally occurring sequence within the modified live Swine Influenza specific vaccine.

In one aspect of the present invention the oligonucleotide probe specific for the modified live Swine Influenza virus specific vaccine binds to a non-naturally occurring sequence within the modified live Swine Influenza specific vaccine within the NS (non-structural protein) gene segment.

The term “NS (non-structural protein)” is known by the person skilled in the art. The segmented genome of influenza A virus consists of eight molecules of linear, negative polarity, single-stranded RNAs which encode eleven polypeptides. The gene segment 8 encodes the two non-structural (NS) proteins, NS1 and NS2.

In one aspect of the present invention the oligonucleotide probe specific for the modified live Swine Influenza virus specific vaccine binds to a non-naturally occurring sequence within the modified live Swine Influenza A specific vaccine between the NS-1 (non-structural protein) and NS-2 ORF.

The term “NS-1 (non-structural protein) and NS-2 ORF” refers to the open reading frame (ORF) NS-1 and NS-2 encoded by gene segment NS of the swine influenza A virus. Gene segment NS of the swine influenza A virus encodes two proteins NS-1 and NS-2.

The term “gene or gene segments” is well known to the person skilled in the art. However, as set forth above influenza A genomes such as the genome of the Swine Influenza virus contains eight gene segments encoding 11 proteins.

In one aspect of the present invention the oligonucleotide probe specific for the modified live Swine Influenza virus specific vaccine is thiolated.

### Oligonucleotide Probe Specific for Swine Influenza Virus

In one aspect of the present invention the oligonucleotide probe specific for the Swine Influenza virus binds to a naturally occurring sequence within the Swine Influenza virus.

In one aspect of the present invention the oligonucleotide probe specific for the Swine Influenza virus is specific for the HA, NA, PB1, PB2, PA, NP, M, or NS gene segment of a Swine Influenza virus.

The term “HA, NA, PB1, PB2, PA, NP, M, or NS gene segment” is described elsewhere herein.

In one aspect of the present invention the oligonucleotide probe specific for the Swine Influenza virus is specific for the NS (non-structural protein) gene segment.

In one aspect of the present invention the oligonucleotide probe specific for the Swine Influenza virus is specific for the NS-1 (non-structural protein-1) ORF.

In one aspect of the present invention the oligonucleotide probe specific for the Swine Influenza virus comprises at least twelve contiguous nucleotides of the sequence shown in SEQ ID NO:11 (gtgtgatctttaaccgattagagactttgt) or SEQ ID NO:13 (TGATACTACTAAGGGCTTTCACTGA) or SEQ ID NO:15 (TGATACTACTAAGAGCTTTCACTGA) or SEQ ID NO:17 (TAATACTACTAAGGGCTTTCACTGA) or SEQ ID NO:19 (TGATACTACTGAGAGCTTTCACTGA) or SEQ ID NO:21 (TGGTACTACTAAGGGCTTTCACTG) or SEQ ID NO:23 (TGATACTACTAAGGGCTTTCACCG) or SEQ ID NO:25 (TGATACTACTGAGGGCTTTCACTG) or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or a sequence having at least 70% sequence identity thereto.

In one aspect of the present invention the oligonucleotide probe specific for the Swine Influenza virus comprises at least twelve, fourteen, sixteen, eighteen, twenty, twenty-two, twenty-four, twenty-six or twenty-eight contiguous nucleotides of the sequence shown in SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or a sequence having at least 70%, at least 75%, at least 80%, at least 85%, at least 90%, at least 95%, at least 97.5% sequence identity thereto.

In one aspect of the present invention the oligonucleotide probe specific for the Swine Influenza virus comprises at least fourteen contiguous nucleotides of the sequence shown in SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or a sequence having at least 70% sequence identity thereto.

In one aspect of the present invention the oligonucleotide probe specific for the Swine Influenza virus comprises at least sixteen contiguous nucleotides of the sequence shown in SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or its complementary reverse sequences or a sequence having at least 70% sequence identity thereto.

In one aspect of the present invention the oligonucleotide probe specific for the Swine Influenza virus comprises at least eighteen contiguous nucleotides of the sequence shown in SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or a sequence having at least 70% sequence identity thereto

In one aspect of the present invention the oligonucleotide probe specific for the Swine Influenza virus comprises at least twenty contiguous nucleotides of the sequence shown in SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or a sequence having at least 70% sequence identity thereto.

In one aspect of the present invention the oligonucleotide probe specific for the Swine Influenza virus comprises at least twenty-two contiguous nucleotides of the sequence shown in SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or a sequence having at least 70% sequence identity thereto.

In one aspect of the present invention the oligonucleotide probe specific for the Swine Influenza virus comprises at least twenty-four contiguous nucleotides of the sequence shown in SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or a sequence having at least 70% sequence identity thereto.

In one aspect of the present invention the oligonucleotide probe specific for the Swine Influenza virus comprises at least twenty-six contiguous nucleotides of the sequence shown in SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or a sequence having at least 70% sequence identity thereto.

In one aspect of the present invention the oligonucleotide probe specific for the Swine Influenza virus comprises at least twenty-eight contiguous nucleotides of the sequence shown in SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or a sequence having at least 70% sequence identity thereto

In one aspect of the present invention the oligonucleotide probe specific for the Swine Influenza virus comprises a sequence shown in SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or a sequence having at least 70% sequence identity thereto.

In one aspect of the present invention said sequence identity of the oligonucleotide probe is at least 80%.

In one aspect of the present invention said sequence identity of the oligonucleotide probe is at least 90%.

In one aspect of the present invention said sequence identity of the oligonucleotide probe is at least 95%.

In one aspect of the present invention said sequence identity of the oligonucleotide probe is at least 97.5%.

In one aspect of the present invention the sequence of the oligonucleotide probe specific for the Swine Influenza virus comprises the sequence shown in SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26).

In one aspect of the present invention the oligonucleotide probe specific for the Swine Influenza virus is thiolated.

### Signal

In one aspect of the present invention the signal is an enzymatic signal, a fluorescent signal or an electrochemical signal.

Fluorescent Label for Oligonucleotide Probe or Primer

In one aspect of the present invention the oligonucleotide probe or primer is coupled with a detectable label selected from the group consisting of a radioactive element and a fluorescent chemical.

In one aspect of the present invention the oligonucleotide probe is coupled with a detectable label selected from the group consisting of a radioactive element and a fluorescent chemical.

In one aspect of the present invention the fluorescent chemical label is selected from a fluorescein, a cyanine dye, a coumarin, a phycoerythrin, a phycobiliprotein, a dansyl chloride, a lanthanide complex or a fluorochrome.

In one aspect of the present invention said fluorochrome is R-phycoerythrin, Cy3, Cy5, Quasar 670, Rhodamin, Alexa, or Texas Red.

In one aspect of the present invention said fluorescein is 6-FAM (6-carboxyfluorescein), TET (6-carboxy-4,7,2′,7′-tetrachlorofluorescein), JOE (2,7-dimethoxy-4,5-dichloro-6-carboxyfluorescein) or HEX (6-carboxy-2′,4′,7′,4,7-hexachlorofluorescein).

Preferably, the oligonucleotide probe of the present invention is labeled with a marker substance for detecting a product amplified by use of the corresponding oligonucleotide primer pair. Preferably, the oligonucleotide probe is coupled with a detectable label selected from the group consisting of a radioactive element, an enzyme, an antibody and a fluorescent chemical. More preferably, the oligonucleotide probe of the present invention is labeled with a fluorescent chemical in order to quickly detect an amplified product with high sensitivity. More preferably, the oligonucleotide probe of the present invention is double-labeled with a fluorescent chemical and a quencher.

Preferably, the oligonucleotide probe of the present invention has the 5′ end modified with a fluorescent substance (reporter fluorescent dye) and the 3′ end modified with a quencher (quenching fluorescent dye) or vice versa. Preferably, the reporter dye is a fluorescein (including 6-FAM (6-carboxyfluorescein), TET (6-carboxy-4,7,2′,7′-tetrachlorofluorescein), JOE (2,7-dimethoxy-4,5-dichloro-6-carboxyfluorescein) and HEX (6-carboxy-2′,4′,7′,4,7-hexachlorofluorescein), a cyanine dye, a coumarin, a phycoerythrin, a phycobiliprotein, a dansyl chloride, a lanthanide complex or a fluorochrome such as R-phycoerythrin, Cy3, Cy5, Quasar 670, rhodamin, Alexa, or Texas Red. Examples of the quenching fluorescent dye include rhodamine type fluorescent dyes such as 6-carboxytetramethylrhodamine (TAMRA), black hole quencher (BHQ) BHQ-1 and 2 and 6-carboxy-X-rhodamine (ROX). However, such labels and techniques are well known to the person skilled in the art and have been extensively described in the literature such as the TagMan assay.

In one aspect of the present invention the oligonucleotide probe is further labeled with a quencher selected from 6-carboxytetramethylrhodamine (TAMRA), black hole quencher (BHQ) BHQ-1 and 2 or 6-carboxy-X-rhodamine (ROX).

In one aspect of the present invention the oligonucleotide probe or primer is coupled with a fluorescent label.

In one aspect of the present invention the method is a qPCR.

### Enzymatic and Electrochemical Label for Oligonucleotide Probes or Primers

In one aspect of the present invention the oligonucleotide probe or primer is coupled with a first coupling group.

In one aspect of the present invention the primer is coupled with a first coupling group.

In one aspect of the present invention the generation of a signal comprises providing a second coupling group.

In one aspect of the present invention said first and second coupling groups are selected from the group consisting of antibody-antigen, receptor-ligand, biotin-streptavidin, sugar-lectins, and complementary oligonucleotides.

In one aspect of the present invention the second coupling group is labelled.

Preferably, the oligonucleotide probe or primer is labelled with biotin and a labelled streptavidin is used for generating the signal.

In one aspect of the present invention the label is selected from the group consisting of a radioactive element, a fluorescent chemical or an enzyme.

In one aspect of the present invention said fluorescent chemical label is a fluorescent as described herein.

In one aspect of the present invention said enzyme label is selected from horseradish peroxidase (HRP), esterase, alkaline phosphatase (AP), Glucose oxidase, β-galactosidase or Luciferase.

Enzymatic labels are well known to the person skilled in the art and any enzymatic assay can be done without further ado. Substrates are well known to the person skilled in the art as well and exemplary comprise 3,3′-diaminobenzidine (DAB), 3,3′,5,5′-tetramethylbenzidine (TMB), 2,2′-Azinobis [3-ethylbenzothiazoline-6-sulfonic acid] (ABTS) or o-phenylenediamine dihydrochloride (OPD) for HRP, combination of nitro blue tetrazolium chloride (NBT) and 5-bromo-4-chloro-3-indolyl phosphate (BCIP) or p-Nitrophenyl Phosphate (PNPP) or p-aminophenol (PAP) for AP, nitro blue tetrazolium chloride (NBT) for Glucose oxidase and 5-bromo-4-chloro-3-indoyl-β-D-galactopyranoside (BCIG or X-Gal) for β-galactosidase.

In one aspect of the present invention the oligonucleotide probe or primer signal is an enzymatic signal.

Preferably, the oligonucleotide probe or primer is labelled with biotin and a streptavidin labelled with alkaline phosphatase (AP) is used for generating the signal.

In one aspect of the present invention said enzyme converts a substrate into a reversible redox couple.

In general, the redox cycling is an electrochemical process in which a molecule is reversibly oxidised and/or reduced (i.e. a redox-active molecule; a redox couple) between at least two electrodes generating a current flow (an electrochemical signal). However, methods and techniques in this respect are well known in the art.

Examples for substrates/redox couples are well known to the person skilled in the art. However, suitable examples include, but are not limited to, ferrocene derivatives, ferrocinium derivatives, mixtures of ferrocene derivatives and ferrocinium derivatives, cupric chloride, cuprous chloride, mixtures of cupric chloride and cuprous chloride, ruthenium-tris-bipyridine, potassium ferrohexacyanide, potassium ferrihexacyanide, and mixtures of potassium ferrohexacyanide and potassium ferrihexacyanide, porphyrinic macrocycle, a metallocene, a linear polyene, a cyclic polyene, a heteroatom-substituted linear polyene, a heteroatom-substituted cyclic polyene, a tetrathiafulvalene, a tetraselenafulvalene, a metal coordination complex, a buckyball, a triarylamine, a 1,4-phenylenediamine, a xanthene, a flavin, a phenazine, a phenothiazine, an acridine, a quinoline, a 2,2′-bipyridyl, a 4,4′-bipyridyl, a tetrathiotetracene, and a pen-bridged naphthalene dichalcogenide.

Preferably, the substrate is a redox molecule having a phosphate group. More preferably, the substrate is a redox molecule having a pyrophosphate. The action of a phosphatase removes the pyrophosphate from the redox molecule. Applicable phosphatase enzymes include, for example alkaline phosphatase, acid phosphatase, protein phosphatase, polyphosphate phosphatase, sugar-phosphatase and pyrophosphatase.

In one aspect of the present invention the substrate is p-aminophenolphosphat.

In one aspect of the present invention the redox couple is p-aminophenol (PAP) and quinoneimine.

Redox cycling techniques comprise Chip technologies such as exemplary the CMOS Chip technology. The CMOS Chip technology has been well described in the prior art. Exemplary, WO 2018/065104 A1, Roland Thewes (Enabling CMOS-based DNA array chips) and Frey et al 2005 (A Digital CMOS DNA Chip) describe the CMOS technology. In general, the electrochemical principle behind this is an enzyme-label-based, current-generation process, so that hybridization of complementary DNA strands translates into sensor currents at the sensor electrodes between 1 pA to 100 nA. Probe molecules (such as the oligonucleotide probes described herein) are immobilized on the surface of a sensor element. The amplification product tagged by an enzyme label (by using alkaline phosphatase labelled primers) is applied to the chip. After the hybridization and washing phases, a chemical substrate (para-aminophenyl-phosphate) is applied to the chip. The enzyme label, available at the sites where hybridization occurred, cleaves the phosphate group and the electrochemically active para-aminophenol is generated. Simultaneously applying an oxidation and a reduction potential to the sensor electrodes, para-aminophenol is oxidized to quinoneimine at the one electrode, and quinoneimine is reduced to para-aminophenol at the other one.

In one aspect of the present invention the oligonucleotide probe or primer signal is an electrochemical signal.

In one aspect of the present invention the method is a DNA Chip based technology.

In one aspect of the present invention the method is CMOS based technology.

### Amplification

In one aspect of the present invention said amplification of polynucleotides is PCR (polymerase chain reaction) or real time PCR (polymerase chain reaction).

Preferably, when using a real time PCR, a calibration curve is prepared using “standards,” which are samples containing a known number of copies of a target nucleic acid sequence. Independent reactions are performed, each containing a different standard. A graph or a “standard curve” of CT vs. Log N (starting copy number) is prepared using CT values from each of the reactions that involved a different amount of standard.

The number of copies of target nucleic acid sequence in a biological sample is determined by interpolating CT values from a reaction containing the biological sample onto the standard curve. Preferably, a software program generates a “standard curve” of CT vs. Log N (starting copy number) for all “standards” and then determines the starting copy number of unknowns by interpolation. The determination of the number of copies of a target gene sequence in a test sample indicates the number of viruses or viral remnants in the test sample.

Such real time PCR method requires at least three oligonucleotides for the analysis of each target nucleic acid sequence. The sequences of forward and reverse oligonucleotide primers are complementary to the ends of the target nucleic acid sequence. A probe sequence is complementary to the sequence found between the ends of the target nucleic acid sequence. A “forward primer” and a “reverse primer” provide a template for polymerase-catalyzed amplification of the target nucleic acid sequence, when hybridized to the target. A single-stranded oligonucleotide probe is required for target detection. In such a method two reactions are combined into a single reaction format: oligonucleotide probe hybridization to detect a specific target nucleic acid sequence and PCR to amplify a target nucleic acid sequence. Through fluorescence resonance energy transfer, the quencher reduces the fluorescence emission of the fluorescent reporter group within the oligonucleotide probe. The 5′-3′ exonuclease activity of Taq Polymerase cleaves the quencher moiety from the bound oligonucleotide probe as it catalyzes complementary strand synthesis, causing the fluorescence emission of the probe to increase, since the reporter is no longer quenched. An increase in the fluorescent signal during the amplification reaction thus depends on the hybridization of both the fluorogenic oligonucleotide probe and oligonucleotide primers to the target sequence. Target discrimination is enhanced because spurious amplification caused by non-specific primer hybridization is not detected. (Lee et al., 1993, Nucleic Acids Res. 21: 3761-3766).

Preferably, multiplex formats are employed to detect more than one target nucleic acid sequence in a single reaction. For example, primers for more than one target gene or primers for different locations on the same target gene, with the corresponding target-specific probes linked to different fluorescent reporters, can detect multiple targets in a single reaction. More preferably, primers for one target gene, with two corresponding target-specific oligonucleotide probes linked to different fluorescent reporters are used to detect multiple targets in a single reaction.

### Primer Pairs

In one aspect of the present invention said forward and said reverse-oligonucleotide primer is specific for the NS (non-structural protein) gene segment.

In one aspect of the present invention said forward-oligonucleotide primer is specific for the NS-1 (non-structural protein-1) ORF.

In one aspect of the present invention said reverse-oligonucleotide primer is specific for the NS-2 (non-structural protein-2) ORF.

In one aspect of the present invention said forward and said reverse-oligonucleotide primer specific for the NS (non-structural protein) gene segment comprise at least twelve contiguous nucleotides of the sequence shown in SEQ ID NO:1 (gataataggctctctttgtg) or SEQ ID NO:2 (aggtaatggtgaaatttctc) or SEQ ID NO:27 to SEQ ID NO:38 or a sequence having at least 70% sequence identity thereto.

In one aspect of the present invention said forward and said reverse-oligonucleotide primer specific for the NS (non-structural protein) gene segment comprise at least twelve, fourteen, sixteen or eighteen contiguous nucleotides of the sequence shown in SEQ ID NO:1; SEQ ID NO:2 or SEQ ID NO:27 to SEQ ID NO:38 or a sequence having at least 70%, at least 75%, at least 80%, at least 85%, at least 90%, at least 95%, at least 97.5% sequence identity thereto.

In one aspect of the present invention said forward and said reverse-oligonucleotide primer specific for the NS (non-structural protein) gene segment comprise at least fourteen contiguous nucleotides of the sequence shown in SEQ ID NO:1; SEQ ID NO:2 or SEQ ID NO:27 to SEQ ID NO:38 or a sequence having at least 70% sequence identity thereto.

In one aspect of the present invention said forward and said reverse-oligonucleotide primer specific for the NS (non-structural protein) gene segment comprise at least sixteen contiguous nucleotides of the sequence shown in SEQ ID NO:1; SEQ ID NO:2 or SEQ ID NO:27 to SEQ ID NO:38 or a sequence having at least 70% sequence identity thereto.

In one aspect of the present invention said forward and said reverse-oligonucleotide primer specific for the NS (non-structural protein) gene segment comprise at least eighteen contiguous nucleotides of the sequence shown in SEQ ID NO:1; SEQ ID NO:2 or SEQ ID NO:27 to SEQ ID NO:38 or a sequence having at least 70% sequence identity thereto.

In one aspect of the present invention said forward and said reverse-oligonucleotide primer specific for the NS (non-structural protein) gene segment comprise the sequence shown in SEQ ID NO:1; SEQ ID NO:2 or SEQ ID NO:27 to SEQ ID NO:38 or a sequence having at least 70% sequence identity thereto.

In one aspect of the present invention said sequence identity of the oligonucleotide primer is at least 80%.

In one aspect of the present invention said sequence identity of the oligonucleotide primer is at least 90%.

In one aspect of the present invention said sequence identity of the oligonucleotide primer is at least 95%.

In one aspect of the present invention said sequence identity of the oligonucleotide primer is at least 97.5%.

In one aspect of the present invention said forward and said reverse-oligonucleotide primer specific for the NS (non-structural protein) gene segment comprise the sequence shown in SEQ ID NO:1; SEQ ID NO:2 or SEQ ID NO:27 to SEQ ID NO:38.

In one aspect of the present invention the oligonucleotide primer is biotinylated.

### Animal

In one aspect of the present invention said animal is swine.

### Sample

In one aspect of the present invention the sample is a nasal sample, oral fluid sample, respiratory tissue sample or lung sample. Preferably, the sample is a nasal sample or oral fluid sample. Preferably, the sample is taken from a pig or piglet being between 1 day and 10 weeks of age, more preferably between 1 day and 6 weeks of age. Preferably, the sample is taken from a pig or piglet that was vaccinated with the Swine Influenza virus specific vaccine 1 day to 15 days before taken the sample.

In one aspect of the present invention the environmental Sample is an air filter sample or a sample of a rope for collecting oral fluid.

### Concentration Sample

In one aspect of the present invention the concentration of the modified live Swine Influenza virus specific vaccine or the Swine Influenza virus is between 2 to 12 log EID50.

In one aspect of the present invention the concentration of the modified live Swine Influenza virus specific vaccine or the Swine Influenza virus is between 4 to 10 log EID50.

In one aspect of the present invention the concentration of the modified live Swine Influenza virus specific vaccine or the Swine Influenza virus is between 6 to 8 log EID50.

### Modified Live Swine Influenza Specific Vaccine

In one aspect of the present invention the modified live Swine Influenza virus specific vaccine comprises a sequence which is identical or complementary to the oligonucleotide probe specific for the modified live Swine Influenza virus specific vaccine as described herein.

In one aspect of the present invention said identical or complementary sequence as described herein is a non-naturally occurring sequence within the modified live Swine Influenza virus specific vaccine.

In one aspect of the present invention said identical or complementary sequence as described herein is within the NS (non-structural protein) gene segment of the modified live Swine Influenza virus specific vaccine.

In one aspect of the present invention said identical or complementary sequence as described herein is between the NS-1 (non-structural protein) and NS-2 ORF of the modified live Swine Influenza virus specific vaccine.

In one aspect of the present invention the modified live Swine Influenza virus specific vaccine is attenuated.

The term “attenuated” refers to a pathogen having a reduced virulence. In the present invention “attenuation” is synonymous with “avirulent”. In the present invention, an attenuated SIV is one in which the virulence has been reduced so that it does not cause clinical signs of a swine influenza infection but is capable of inducing an immune response in the target mammal, but may also mean that the clinical signs are reduced in incidence or severity in animals infected with the attenuated SIV in comparison with a “control group” of animals infected with non-attenuated SIV and not receiving the attenuated virus. In this context, the term “reduce/reduced” means a reduction of at least 10%, preferably 25%, even more preferably 50%, still more preferably 60%, even more preferably 70%, still more preferably 80%, still more preferably 90%, even more preferably 95% and most preferably of 100% as compared to the control group as defined above. Thus, an attenuated, avirulent SIV strain is one that suitable for incorporation into an immunogenic composition comprising a modified live SIV.

Preferably, the term “attenuated”, as mentioned herein, is particularly directed to a genetically engineered change in a genomic sequence, such as by truncation of the NS1 gene or protein, which in particular results in a virus growing to titers significantly lower than wild type swine influenza virus in the infected host, when propagated under the same conditions and/or having defective IFN antagonist activity.

In one aspect of the present invention the modified live Swine Influenza virus specific vaccine is bivalent.

In one aspect of the present invention the modified live Swine Influenza virus specific vaccine comprises modified live H3N2 and H1N1 Swine Influenza virus.

In one aspect of the present invention the modified live H3N2 and H1N1 viruses of swine influenza virus have a deletion within the NS1 gene.

Further, the term “deletion within the NS1 gene” means that one or more amino acids are deleted within the NS1 protein and one or more nucleic acids are deleted within the NS1 ORF or nucleotide sequence, respectively. However, the term NS1 does not refer to the NS1 ORF only, but also refers to NS1 ORF products (such as RNA or protein) encoded by the NS1 ORF. In the case of a protein, the NS1 gene product is full-length and has wild-type NS1 activity, (e.g., from Influenza A/swine/Texas/4199-2/98). The full length wildtype swine NS1 proteins vary between 217 to 237 amino acids. However, in most cases the full length wildtype swine NS1 protein is 219 amino acids. Representative swine NS1 genes can be found in public sequence databases such as Genbank and include, but are not limited to, Genbank Accession No. AJ293939 (A/swine/Italy/13962/95(H3N2)) and Genbank Accession No. AJ344041 (A/swine/Cotes d'Armor/1121/00(H1N1)).

The term “H1N1” and “H3N2” is known by the person skilled in the art. However, in general, type A influenza viruses are divided into 17 HA (hemagglutinin) and 10 NA (Neuraminidase) subtypes which can give rise to many possible combinations (designated as H1N1, H1N2 . . . H2N1, H2N2 . . . H5N1, H5N2 . . . and so on). Thus, the terms “H1N1” and “H3N2” refer to a specific combination of hemagglutinin (HA) and neuraminidase (NA) subtypes of the SIV.

In one aspect of the present invention the modified live H3 and H1 viruses of swine influenza have a carboxy-terminal truncated NS1 protein.

The term “carboxy-terminal truncated” refers to the truncation of the NS1 protein of the carboxy terminus. The term “carboxy terminus” already has been described above. The term “truncated or truncation” refers to the deletion of one or more amino acid within the NS1 protein or the deletion of one or more nucleic acids within the NS1 gene or nucleotide sequence. Thus, portions of the amino terminal region of the NS1 gene product are retained whereas portions of the carboxy terminus region of the NS1 gene product are deleted.

The term “amino acid sequence”, “polypeptide” and “protein” are used interchangeable. The term “amino acid sequence” refers to a sequence of amino acids composed of the natural occurring amino acids as well as derivatives thereof. The naturally occurring amino acids are well known in the art and are described in standard text books of biochemistry. Within the amino acid sequence the amino acids are connected by peptide bonds. Further, the two ends of the amino acid sequence are referred to as the carboxyl terminus (C-terminus) and the amino terminus (N-terminus).

Preferably, the attenuated swine influenza virus of the invention comprises a genome comprising a mutation in the NS1 gene resulting in a deletion consisting of 5, preferably 10, 15, 20, 25, 30, 35, 40, 45, 50, 55, 60, 65, 70, 75, 80, 85, 90, 91, 92, 93, 94, 95, 100, 105, 110, 115, 119, 120, 121, 125, 130, 135, 140, 145, 146, 147, 148, 150, 155, 160, 165, 170 or 175 amino acid residues from the carboxy terminus of NS1 or a deletion of between 5-170, 25-170, 50-170, 100-170, 90-160, 100-160 or 105-160, 90-150, 5-75, 5-50 or 5-25 amino acid residues from the carboxy terminus.

More preferably, the attenuated swine influenza virus of the invention comprises a genome comprising a mutation in the NS1 gene resulting in a deletion of all amino acid residues of the NS1 gene product except amino acid residues 1-130, amino acid residues 1-129, amino acid residues 1-128, amino acid residues 1-127, amino acid residues 1-126, amino acid residues 1-125, amino acid residues 1-124, amino acid residues 1-123, amino acid residues 1-122, amino acid residues 1-121, amino acid residues 1-120, amino acid residues 1-115, amino acid residues 1-110, amino acid residues 1-100, amino acid residues 1-99, amino acid residues 1-95, amino acid residues 1-85, amino acid residues 1-80, amino acid residues 1-75, amino acid residues 1-73, amino acid residues 1-70, amino acid residues 1-65 or amino acid residues 1-60, wherein the amino terminal amino acid is number 1.

In one aspect of the present invention the modified live H3N2 and H1N1 viruses of swine influenza virus encode for a carboxy-terminal truncated NS1 protein comprising NS1 amino acids 1 through 124, 1 through 125, 1 through 126, 1 through 127 or 1 through 128, wherein the amino terminal amino acid is number 1.

The term “carboxy terminus” or “carboxy-terminal” is well known to the person skilled in the art. The carboxy terminus is also termed carboxyl-terminus, C-terminus, C-terminal tail, C-terminal end, or COOH-terminus. When the protein is translated from messenger RNA, it is created from N-terminus to C-terminus. Thus, the carboxy terminus is the end of an amino acid chain (protein or polypeptide), terminated by a free carboxyl group (—COOH).

In one aspect of the present invention the modified live H3N2 and H1N1 viruses of swine influenza virus encode for a carboxy-terminal truncated NS1 protein comprising NS1 amino acids 1 through 126, wherein the amino terminal amino acid is number 1.

In one aspect of the present invention the modified live H3N2 and H1N1 viruses of swine influenza virus have a carboxy-terminal truncated NS1 protein resulting in a deletion of 91, 92, 93 or 94 amino acid residues from the carboxy terminus of NS1.

In one aspect of the present invention the modified live H3N2 and H1N1 viruses of swine influenza virus have a NS1 gene or protein from A/Swine/Texas/4199-2/98.

In one aspect of the present the modified live H3N2 virus of swine influenza is TX/98/del 126.

The term “TX/98/del 126” refers to the A/Swine/Texas/4199-2/98 strain having a NS1 deletion mutant encoding for a carboxy-terminal truncated NS1 protein comprising of NS1 amino acids 1 through 126, wherein the amino terminal amino acid is number 1.

In one aspect of the present invention the modified live H1N1 virus of swine influenza contains HA and NA from A/swine/Minnesota/37866/1999 (H1N1) and PB2, PB1, PA, NP, M from A/Swine/Texas/4199-2/98 (H3N2) and the NS1-126 gene is from A/Swine/Texas/4199-2/98 (H3N2).

In one aspect of the present invention the modified live H1 virus of swine influenza is a chimeric of A/swine/Minnesota/37866/1999 and TX/98/del 126.

In one aspect of the present invention the modified live H3N2 virus of swine influenza is TX/98/del 126 containing the HA, NA, PB2, PB1, PA, NP, and M from A/Swine/Texas/4199-2/98 and the NS1-126 gene is from A/Swine/Texas/4199-2/98 and, wherein the modified live H1N1 virus of swine influenza contains HA and NA from A/swine/Minnesota/37866/1999 (H1N1) and PB2, PB1, PA, NP, M from A/Swine/Texas/4199-2/98 (H3N2) and the NS1-126 gene is from A/Swine/Texas/4199-2/98 (H3N2).

The term “HA, NA, PB2, PB1, PA, NP, and M” refers to the gene segments or genes of the swine influenza virus. In general, influenza A genomes contain eight gene segments encoding 11 proteins. These proteins include the RNA-dependent RNA polymerase proteins (PB2, PB1 and PA) and nucleoprotein (NP) which form the nucleocapsid; the matrix membrane proteins (M1, M2); two surface glycoproteins which project from the lipid containing envelope: hemagglutinin (HA) and neuraminidase (NA); the nonstructural protein (NS1), nuclear export protein (NEP); and the proapoptotic factor PB1-F2.

In one aspect of the present invention the modified live H3 virus of swine influenza is the H3N2 NS1 deletion mutant of swine influenza virus described in WO 2006/083286 A2 designated as TX/98/de1126.

In one aspect of the present invention the modified live Swine Influenza virus specific vaccine is the bivalent vaccine as described in WO 2016/137929 A1 or the vaccine Ingelvac Provenza™. In particular, the bivalent vaccine is described in paragraph 15 to 140 of WO 2016/137929 A1 or Example 1 of WO 2016/137929 A1.

### Kit

In one aspect of the present invention said at least one forward and one reverse-oligonucleotide primer pair and said oligonucleotide probe specific for the modified live Swine Influenza virus specific vaccine are in one container.

In one aspect of the present invention said at least one forward and one reverse-oligonucleotide primer pair and said oligonucleotide probe specific for the modified live Swine Influenza virus specific vaccine are in two or more separate containers.

In one aspect of the present invention said at least one forward and one reverse-oligonucleotide primer pair, said oligonucleotide probe specific for the modified live Swine Influenza virus specific vaccine and said oligonucleotide probe specific for the Swine Influenza virus are in one container.

In one aspect of the present invention said at least one forward and one reverse-oligonucleotide primer pair, said oligonucleotide probe specific for the modified live Swine Influenza virus specific vaccine and said oligonucleotide probe specific for the Swine Influenza virus are in two or more separate containers.

In one aspect of the present invention said oligonucleotide probe specific for the modified live Swine Influenza virus specific vaccine and said oligonucleotide probe specific for the Swine Influenza virus are in one container.

In one aspect of the present invention said oligonucleotide probe specific for the modified live Swine Influenza virus specific vaccine and said oligonucleotide probe specific for the Swine Influenza virus are in two or more separate containers.

In one aspect of the present invention said kit comprises one or more control samples.

In one aspect of the present invention said control sample is a RNA, cDNA or DNA sample.

In one aspect of the present invention the control is a positive control comprising RNA, cDNA or DNA specific for the modified live Swine Influenza virus specific vaccine.

In one aspect of the present invention the control is a positive control comprising RNA, cDNA or DNA specific for the Swine Influenza virus.

In one aspect of the present invention said kit comprises an instruction letter providing information for use of the kit.

Sequences Overview:

### The Following Sequences are Detailed and Disclosed Hereby in the Present Invention:

NSfor Primer
SEQ ID NO: 1
(gataataggctctctttgtg)
NSrev Primer
SEQ ID NO: 2
(aggtaatggtgaaatttctc)
MLVfluprobe
SEQ ID NO: 3
tagatcttgattaattaa (18 nt): oligonucleotide probe
specific for the modified live Swine Influenza
specific vaccine (“core sequence”)
SEQ ID NO: 4
ttaattaatcaagatcta (18 nt): reverse complement
sequence of SEQ ID NO: 3
MLVfluprobe
SEQ ID NO: 5
(agtagatcttgattaattaagagggagc)
SEQ ID NO: 6
gctccctcttaattaatcaagatctact: reverse complement
sequence of SEQ ID NO: 5
MLVprobe
SEQ ID NO: 7
(atggaaaagtagatcttgattaattaagagg)
SEQ ID NO: 8
cctcttaattaatcaagatctacttttccat: reverse
complement sequence of SEQ ID NO: 7
MLVprobe
SEQ ID NO: 9
(agtagatcttgattaattaagagggagcaatcg)
SEQ ID NO: 10
cgattgctccctcttaattaatcaagatctact: reverse
complement sequence of SEQ ID NO: 9
WTfluprobe
SEQ ID NO: 11
(gtgtgatctttaaccgattagagactttg)
SEQ ID NO: 12
caaagtctctaatcggttaaagatcacac: reverse complement
sequence of SEQ ID NO: 11
WTfluprobe
SEQ ID NO: 13
(TGATACTACTAAGGGCTTTCACTGA)
SEQ ID NO: 14
TCAGTGAAAGCCCTTAGTAGTATCA: reverse complement
sequence of SEQ ID NO: 13
WTfluprobe
SEQ ID NO: 15
(TGATACTACTAAGAGCTTTCACTGA)
SEQ ID NO: 16
TCAGTGAAAGCTCTTAGTAGTATCA: reverse complement
sequence of SEQ ID NO:15
WTfluprobe
SEQ ID NO: 17
(TAATACTACTAAGGGCTTTCACTGA)
SEQ ID NO: 18
TCAGTGAAAGCCCTTAGTAGTATTA: reverse complement
sequence of SEQ ID NO: 17
WTfluprobe
SEQ ID NO: 19
(TGATACTACTGAGAGCTTTCACTGA)
SEQ ID NO: 20
TCAGTGAAAGCTCTCAGTAGTATCA: reverse complement
sequence of SEQ ID NO: 19
WTfluprobe
SEQ ID NO: 21
(TGGTACTACTAAGGGCTTTCACTG)
SEQ ID NO: 22
CAGTGAAAGCCCTTAGTAGTACCA: reverse complement
sequence of SEQ ID NO: 21
WTfluprobe
SEQ ID NO: 23
(TGATACTACTAAGGGCTTTCACCG)
SEQ ID NO: 24
CGGTGAAAGCCCTTAGTAGTATCA: reverse complement
sequence of SEQ ID NO: 23
WTfluprobe
SEQ ID NO: 25
(TGATACTACTGAGGGCTTTCACTG)
SEQ ID NO: 26
CAGTGAAAGCCCTCAGTAGTATCA: reverse complement
sequence of SEQ ID NO: 25
NSfor Primer
SEQ ID NO: 27
(GATAATAGGCCCTCTTTGTG)
NSfor Primer
SEQ ID NO: 28
(GATAATAGGCCCTCTTTGC)
NSfor Primer
SEQ ID NO: 29
(GATAACAGGCTCTCTTTGTG)
NSfor Primer
SEQ ID NO: 30
(CAATAGGCCCTCTTTGTG)
NSfor Primer
SEQ ID NO: 31
(GATAATAGGCTTTCTTTGTGTG)
NSrev Primer
SEQ ID NO: 32
(AGGCAATGGTGAAATTTCTC)
NSrev Primer
SEQ ID NO: 33
(AAGGTAATGATGAAATTTCTCC)
NSrev Primer
SEQ ID NO: 34
(AGGTAATGGTGAAATTTCAC)
NSrev Primer
SEQ ID NO: 35
(AGGTAATGGTGAGATTTCTC)
NSrev Primer
SEQ ID NO: 36
(AGGTAAGGGTGAAATTTCTC)
NSfor Primer
SEQ ID NO: 37
(gataataggctctctttgtgtgc)
NSrev Primer
SEQ ID NO: 38
(gagaaggtaatggtgaaatttctc)
CMOS Thiol Probes
SEQ ID NO: 39
AGTAGATCTTGATTAATTAAGAGGGAGCAATCG
SEQ ID NO: 40
CGATTGCTCCCTCTTAATTAATCAAGATCTACT: reverse
complement sequence of SEQ ID NO: 39

### DETAILED DESCRIPTION

The following Clauses are described herein:
1. A diagnostic kit for the detection of an animal vaccinated with a modified live Swine Influenza virus specific vaccine comprising an oligonucleotide probe specific for the modified live Swine Influenza virus specific vaccine comprising at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto.
2. A diagnostic kit for differentiating animals vaccinated with a modified live Swine Influenza virus specific vaccine from animals infected with Swine Influenza virus comprising
   a. an oligonucleotide probe specific for the modified live Swine Influenza virus specific vaccine comprising at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto;
   b. an oligonucleotide probe specific for the Swine Influenza virus for detecting an infection with Swine Influenza virus.
3. A method for detecting an animal vaccinated with a modified live Swine Influenza virus specific vaccine in a biological sample comprising the steps of:
   a. obtaining a biological sample containing at least one nucleic acid from an animal;
   b. providing a forward and a reverse-oligonucleotide primer pair and an oligonucleotide probe specific for the modified live Swine Influenza virus specific vaccine, said oligonucleotide probe comprises at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto;
   c. contacting said oligonucleotide primer pair with said biological sample under conditions which allow for amplification of polynucleotides;
   d. generating a signal using said oligonucleotide probe specific for the modified live Swine Influenza virus specific vaccine; and
   e. detecting said signal, wherein detection of said signal indicates a vaccination with a Swine Influenza virus specific vaccine in the biological sample.
4. A method for detecting an animal vaccinated with a modified live Swine Influenza virus specific vaccine in a biological sample comprising the steps of:
   a. obtaining a biological sample containing at least one nucleic acid from an animal;
   b. providing a forward and a reverse-oligonucleotide primer pair and an oligonucleotide probe specific for the modified live Swine Influenza virus specific vaccine, said oligonucleotide probe comprises at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto;
   c. contacting said oligonucleotide primer pair and said oligonucleotide probe with said biological sample under conditions which allow for amplification of polynucleotides;
   d. generating an amplification product and an oligonucleotide probe signal; and
   e. detecting said oligonucleotide probe signal, wherein detection of the oligonucleotide probe signal indicates a vaccination with a Swine Influenza virus specific vaccine in the biological sample.
5. A method of differentiating animals vaccinated with a modified live Swine Influenza virus specific vaccine from animals infected with Swine Influenza virus, comprising
   a. obtaining a biological sample containing at least one nucleic acid from an animal;
   b. providing
      i) at least one forward and one reverse-oligonucleotide primer pair, and,
      ii) an oligonucleotide probe specific for the modified live Swine Influenza virus specific vaccine for detecting a vaccination with a Swine Influenza virus specific vaccine, said oligonucleotide probe comprises at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto, and,
      iii) an oligonucleotide probe specific for the Swine Influenza virus for detecting an infection with Swine Influenza virus;
   c. contacting said oligonucleotide primer pair with said biological sample under conditions which allow for amplification of polynucleotides;
   d. generating a signal using said oligonucleotide probe specific for the modified live Swine Influenza specific vaccine and/or said oligonucleotide probe specific for the Swine Influenza virus; and
   e. detecting said signal, wherein
      i) detection of a signal using said oligonucleotide probe specific for the modified live Swine Influenza specific vaccine indicates a vaccination with a Swine Influenza specific vaccine in the biological sample, and,
      ii) detection of signal using said oligonucleotide probe specific for the Swine Influenza virus indicates an infection with a Swine Influenza virus in the biological sample.
6. A method of differentiating animals vaccinated with a modified live Swine Influenza virus specific vaccine from animals infected with Swine Influenza virus, comprising
   a. obtaining a biological sample containing at least one nucleic acid from an animal;
   b. providing
      i) at least one forward and one reverse-oligonucleotide primer pair, and,
      ii) an oligonucleotide probe specific for the modified live Swine Influenza virus specific vaccine for detecting a vaccination with a Swine Influenza virus specific vaccine, said oligonucleotide probe comprises at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto, and,
      iii) an oligonucleotide probe specific for the Swine Influenza virus for detecting an infection with Swine Influenza virus;
   c. contacting said oligonucleotide primer pair and said oligonucleotide probes with said biological sample under conditions which allow for amplification of polynucleotides;
   d. generating an amplification product and an oligonucleotide probe signal; and
   e. detecting said oligonucleotide probe signal, wherein
      i) detection of an oligonucleotide probe signal from the oligonucleotide probe specific for the modified live Swine Influenza virus specific vaccine indicates a vaccination with a Swine Influenza specific vaccine in the biological sample, and,
      ii) detection of an oligonucleotide probe signal from the oligonucleotide probe specific for the Swine Influenza virus indicates an infection with a Swine Influenza virus in the biological sample.
7. A method for detecting animals vaccinated with a modified live Swine Influenza virus specific vaccine within a group of animals comprising the steps of:
   a. obtaining an environmental Sample containing at least one nucleic acid from an animal;
   b. providing a forward and a reverse-oligonucleotide primer pair and an oligonucleotide probe specific for the modified live Swine Influenza virus specific vaccine, said oligonucleotide probe comprises at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto;
   c. contacting said oligonucleotide primer pair and said oligonucleotide probe with said environmental Sample under conditions which allow for amplification of polynucleotides;
   d. generating a signal using said oligonucleotide probe specific for the modified live Swine Influenza specific vaccine; and
   e. detecting the oligonucleotide probe signal, wherein the presence of the oligonucleotide probe signal indicates a vaccination with the Swine Influenza virus specific vaccine within said group of animals.
8. A method for detecting animals vaccinated with a modified live Swine Influenza virus specific vaccine within a group of animals comprising the steps of:
   a. obtaining an environmental Sample containing at least one nucleic acid from an animal;
   b. providing a forward and a reverse-oligonucleotide primer pair and an oligonucleotide probe specific for the modified live Swine Influenza virus specific vaccine, said oligonucleotide probe comprises at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto;
   c. contacting said oligonucleotide primer pair and said oligonucleotide probe with said environmental Sample under conditions which allow for amplification of polynucleotides;
   d. generating an amplification product and an oligonucleotide probe signal; and
   e. detecting the oligonucleotide probe signal, wherein the presence of the oligonucleotide probe signal indicates a vaccination with the Swine Influenza virus specific vaccine within said group of animals.
9. A method for determining a ratio between animals vaccinated with a modified live Swine Influenza virus specific vaccine and animals infected with Swine Influenza virus within a group of animals comprising the steps of:
   a. obtaining an environmental Sample containing at least one nucleic acid from an animal;
   b. providing
      i) at least one forward and one reverse-oligonucleotide primer pair, and,
      ii) an oligonucleotide probe specific for the modified live Swine Influenza virus specific vaccine for detecting a vaccination with a Swine Influenza virus specific vaccine, said oligonucleotide probe comprises at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto, and,
      iii) an oligonucleotide probe specific for the Swine Influenza virus for detecting an infection with Swine Influenza virus;
   c. contacting said oligonucleotide primer pair and said oligonucleotide probes with said environmental Sample under conditions which allow for amplification of polynucleotides;
   d. generating a signal using said oligonucleotide probe specific for the modified live Swine Influenza specific vaccine and/or said oligonucleotide probe specific for the Swine Influenza virus; and
   e. detecting the oligonucleotide probe signal from
      i) the oligonucleotide probe specific for the modified live Swine Influenza virus specific vaccine, and,
      ii) from the oligonucleotide probe specific for the Swine Influenza virus;

f. generating a ratio of i) and ii) or ii) and i) of step e.
10. A method for determining a ratio between animals vaccinated with a modified live Swine Influenza virus specific vaccine and animals infected with Swine Influenza virus within a group of animals comprising the steps of:
   a. obtaining an environmental Sample containing at least one nucleic acid from an animal;
   b. providing
      i) at least one forward and one reverse-oligonucleotide primer pair, and,
      ii) an oligonucleotide probe specific for the modified live Swine Influenza virus specific vaccine for detecting a vaccination with a Swine Influenza virus specific vaccine, said oligonucleotide probe comprises at least twelve contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto, and,
      iii) an oligonucleotide probe specific for the Swine Influenza virus for detecting an infection with Swine Influenza virus;
   c. contacting said oligonucleotide primer pair and said oligonucleotide probes with said environmental Sample under conditions which allow for amplification of polynucleotides;
   d. generating an amplification product and an oligonucleotide probe signal; and
   e. detecting the oligonucleotide probe signal from
      i) the oligonucleotide probe specific for the modified live Swine Influenza virus specific vaccine, and,
      ii) from the oligonucleotide probe specific for the Swine Influenza virus;
   f. generating a ratio of i) and ii) or ii) and i) of step e.
11. The method of any one of clauses 3 to 10, wherein step a or c comprises extracting said nucleic acid from said biological sample or said environmental Sample.
12. The method of any one of clauses 3 to 11, wherein step a or c comprises a reverse transcription of the RNA.
13. The diagnostic kit according to clause 1 or 2, wherein said kit comprises at least one forward and reverse-oligonucleotide primer pair.
14. The diagnostic kit, method for detecting or method of differentiating of any one of clauses 1 to 13, wherein the oligonucleotide probe specific for the modified live Swine Influenza virus specific vaccine comprises at least fourteen contiguous nucleotides of the sequence shown in SEQ ID NO:3 or its reverse complementary sequence (SEQ ID NO:4) or a sequence having at least 70% sequence identity thereto.
15. The diagnostic kit, method for detecting or method of differentiating of any one of clauses 1 to 14, wherein the oligonucleotide probe specific for the modified live Swine Influenza virus specific vaccine comprises at least fifteen contiguous nucleotides of the sequence shown in SEQ ID NO:3 or its reverse complementary sequence (SEQ ID NO:4) or a sequence having at least 70% sequence identity thereto.
16. The diagnostic kit, method for detecting or method of differentiating of any one of clauses 1 to 15, wherein the oligonucleotide probe specific for the modified live Swine Influenza virus specific vaccine comprises at least sixteen contiguous nucleotides of the sequence shown in SEQ ID NO:3 or its reverse complementary sequence (SEQ ID NO:4) or a sequence having at least 70% sequence identity thereto.
17. The diagnostic kit, method for detecting or method of differentiating of any one of clauses 1 to 16, wherein the oligonucleotide probe specific for the modified live Swine Influenza virus specific vaccine comprises at least seventeen contiguous nucleotides of the sequence shown in SEQ ID NO:3 or its reverse complementary sequence (SEQ ID NO:4) or a sequence having at least 70% sequence identity thereto.
18. The diagnostic kit, method for detecting or method of differentiating of any one of clauses 1 to 17, wherein the oligonucleotide probe specific for the modified live Swine Influenza virus specific vaccine comprises a sequence shown in SEQ ID NO:3 or its reverse complementary sequence (SEQ ID NO:4) or a sequence having at least 70% sequence identity thereto.
19. The diagnostic kit, method for detecting or method of differentiating of any one of clauses 1 to 18, wherein the oligonucleotide probe specific for the modified live Swine Influenza virus specific vaccine comprises at least twelve contiguous nucleotides of the sequence shown in SEQ ID NO:5 (agtagatcttgattaattaagagggagc) or SEQ ID NO 7: (atggaaaagtagatcttgattaattaagagg), SEQ ID NO 9: (agtagatcttgattaattaagagggagcaatcg) or SEQ ID NO: 39 (AGTAGATCTTGATTAATTAAGAGGGAGCAATCG) or its complementary reverse sequences (SEQ ID NO:6; SEQ ID NO:8; SEQ ID NO:10; SEQ ID NO: 40) or a sequence having at least 70% sequence identity thereto.
20. The diagnostic kit, method for detecting or method of differentiating of any one of clauses 1 to 19, wherein the oligonucleotide probe specific for the modified live Swine Influenza virus specific vaccine comprises at least fourteen contiguous nucleotides of the sequence shown in SEQ ID NO:5, SEQ ID NO:7, SEQ ID NO:9 or SEQ ID NO: 39 or its complementary reverse sequences (SEQ ID NO:6; SEQ ID NO:8; SEQ ID NO:10; SEQ ID NO: 40) or a sequence having at least 70% sequence identity thereto.
21. The diagnostic kit, method for detecting or method of differentiating of any one of clauses 1 to 20, wherein the oligonucleotide probe specific for the modified live Swine Influenza virus specific vaccine comprises at least sixteen contiguous nucleotides of the sequence shown in SEQ ID NO:5, SEQ ID NO:7, SEQ ID NO:9 or SEQ ID NO: 39 or its complementary reverse sequences (SEQ ID NO:6; SEQ ID NO:8; SEQ ID NO:10; SEQ ID NO: 40) or a sequence having at least 70% sequence identity thereto.
22. The diagnostic kit, method for detecting or method of differentiating of any one of clauses 1 to 21, wherein the oligonucleotide probe specific for the modified live Swine Influenza virus specific vaccine comprises at least eighteen contiguous nucleotides of the sequence shown in SEQ ID NO:5, SEQ ID NO:7, SEQ ID NO:9 or SEQ ID NO: 39 or its complementary reverse sequences (SEQ ID NO:6; SEQ ID NO:8; SEQ ID NO:10; SEQ ID NO: 40) or a sequence having at least 70% sequence identity thereto.
23. The diagnostic kit, method for detecting or method of differentiating of any one of clauses 1 to 22, wherein the oligonucleotide probe specific for the modified live Swine Influenza virus specific vaccine comprises at least twenty contiguous nucleotides of the sequence shown in SEQ ID NO:5, SEQ ID NO:7, SEQ ID NO:9 or SEQ ID NO: 39 or its complementary reverse sequences (SEQ ID NO:6; SEQ ID NO:8; SEQ ID NO:10; SEQ ID NO: 40) or a sequence having at least 70% sequence identity thereto.
24. The diagnostic kit, method for detecting or method of differentiating of any one of clauses 1 to 23, wherein the oligonucleotide probe specific for the modified live Swine Influenza virus specific vaccine comprises at least twenty-two contiguous nucleotides of the sequence shown in SEQ ID NO:5, SEQ ID NO:7, SEQ ID NO:9 or SEQ ID NO: 39 or its complementary reverse sequences (SEQ ID NO:6; SEQ ID NO:8; SEQ ID NO:10; SEQ ID NO: 40) or a sequence having at least 70% sequence identity thereto.
25. The diagnostic kit, method for detecting or method of differentiating of any one of clauses 1 to 24, wherein the oligonucleotide probe specific for the modified live Swine Influenza virus specific vaccine comprises at least twenty-four contiguous nucleotides of the sequence shown in SEQ ID NO:5, SEQ ID NO:7, SEQ ID NO:9 or SEQ ID NO: 39 or its complementary reverse sequences (SEQ ID NO:6; SEQ ID NO:8; SEQ ID NO:10; SEQ ID NO: 40) or a sequence having at least 70% sequence identity thereto.
26. The diagnostic kit, method for detecting or method of differentiating of any one of clauses 1 to 25, wherein the oligonucleotide probe specific for the modified live Swine Influenza virus specific vaccine comprises at least twenty-six contiguous nucleotides of the sequence shown in SEQ ID NO:5, SEQ ID NO:7, SEQ ID NO:9 or SEQ ID NO: 39 or its complementary reverse sequences (SEQ ID NO:6; SEQ ID NO:8; SEQ ID NO:10; SEQ ID NO: 40) or a sequence having at least 70% sequence identity thereto.
27. The diagnostic kit, method for detecting or method of differentiating of any one of clauses 1 to 26, wherein the oligonucleotide probe specific for the modified live Swine Influenza virus specific vaccine comprises a sequence shown in SEQ ID NO:5, SEQ ID NO:7, SEQ ID NO:9 or SEQ ID NO: 39 or its complementary reverse sequences (SEQ ID NO:6; SEQ ID NO:8; SEQ ID NO:10; SEQ ID NO: 40) or a sequence having at least 70% sequence identity thereto.
28. The diagnostic kit, method for detecting or method of differentiating of any one of clauses 1 to 27, wherein said sequence identity of the oligonucleotide probe is at least 80%.
29. The diagnostic kit, method for detecting or method of differentiating of any one of clauses 1 to 28, wherein said sequence identity of the oligonucleotide probe is at least 90%.
30. The diagnostic kit, method for detecting or method of differentiating of any one of clauses 1 to 29, wherein said sequence identity of the oligonucleotide probe is at least 95%.
31. The diagnostic kit, method for detecting or method of differentiating of any one of clauses 1 to 30, wherein said sequence identity of the oligonucleotide probe is at least 97.5%.
32. The diagnostic kit, method for detecting or method of differentiating of any one of clauses 1 to 31, wherein the sequence of the oligonucleotide probe specific for the modified live Swine Influenza virus specific vaccine comprises the sequence shown in SEQ ID NO:5 or its complementary reverse sequence (SEQ ID NO:6).
33. The diagnostic kit, method for detecting or method of differentiating of any one of clauses 1 to 31, wherein the sequence of the oligonucleotide probe specific for the modified live Swine Influenza virus specific vaccine comprises the sequence shown in SEQ ID NO:7 or its complementary reverse sequence (SEQ ID NO:8).
34. The diagnostic kit, method for detecting or method of differentiating of any one of clauses 1 to 31, wherein the sequence of the oligonucleotide probe specific for the modified live Swine Influenza virus specific vaccine comprises the sequence shown in SEQ ID NO:9 or its complementary reverse sequence (SEQ ID NO:10) or wherein the sequence of the oligonucleotide probe specific for the modified live Swine Influenza virus specific vaccine comprises the sequence shown in SEQ ID NO:39 or its complementary reverse sequence (SEQ ID NO:40).
35. The diagnostic kit, method for detecting or method of differentiating of any one of clauses 1 to 34, wherein the oligonucleotide probe specific for the modified live Swine Influenza virus specific vaccine binds to a non-naturally occurring sequence within the modified live Swine Influenza specific vaccine.
36. The diagnostic kit, method for detecting or method of differentiating of any one of clauses 1 to 35, wherein the oligonucleotide probe specific for the modified live Swine Influenza virus specific vaccine binds to a non-naturally occurring sequence within the modified live Swine Influenza specific vaccine within the NS (non-structural protein) gene segment.
37. The diagnostic kit, method for detecting or method of differentiating of any one of clauses 1 to 36, wherein the oligonucleotide probe specific for the modified live Swine Influenza virus specific vaccine binds to a non-naturally occurring sequence within the modified live Swine Influenza specific vaccine between the NS-1 (non-structural protein) and NS-2 ORF.
38. The diagnostic kit, method for detecting or method of differentiating of any one of clauses 2, 5, 6 and 9 to 37, wherein the oligonucleotide probe specific for the Swine Influenza virus binds to a naturally occurring sequence within the Swine Influenza virus.
39. The diagnostic kit, method for detecting or method of differentiating of any one of clauses 2, 5, 6, and 9 to 38, wherein the oligonucleotide probe specific for the Swine Influenza virus is specific for the HA, NA, PB1, PB2, PA, NP, M, or NS gene segment of a Swine Influenza virus.
40, The diagnostic kit, method for detecting or method of differentiating of any one of clauses 2, 5, 6 and 9 to 39, wherein the oligonucleotide probe specific for the Swine Influenza virus is specific for the NS (non-structural protein) gene segment.
41. The diagnostic kit, method for detecting or method of differentiating of any one of clauses 2, 5, 6 and 9 to 40, wherein the oligonucleotide probe specific for the Swine Influenza virus is specific for the NS-1 (non-structural protein-1) ORF.
42. The diagnostic kit, method for detecting or method of differentiating of any one of clauses 2, 5, 6 and 9 to 41, wherein the oligonucleotide probe specific for the Swine Influenza virus comprises at least twelve contiguous nucleotides of the sequence shown in SEQ ID NO:11 (gtgtgatctttaaccgattagagactttgt) or SEQ ID NO:13 (TGATACTACTAAGGGCTTTCACTGA) or SEQ ID NO:15 (TGATACTACTAAGAGCTTTCACTGA) or SEQ ID NO:17 (TAATACTACTAAGGGCTTTCACTGA) or SEQ ID NO:19 (TGATACTACTGAGAGCTTTCACTGA) or SEQ ID NO:21 (TGGTACTACTAAGGGCTTTCACTG) or SEQ ID NO:23 (TGATACTACTAAGGGCTTTCACCG) or SEQ ID NO:25 (TGATACTACTGAGGGCTTTCACTG) or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or a sequence having at least 70% sequence identity thereto.
43. The diagnostic kit, method for detecting or method of differentiating of any one of clauses 2, 5, 6 and 9 to 42, wherein the oligonucleotide probe specific for the Swine Influenza virus comprises at least fourteen contiguous nucleotides of the sequence shown SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or a sequence having at least 70% sequence identity thereto.
44. The diagnostic kit, method for detecting or method of differentiating of any one of clauses 2, 5, 6 and 9 to 43, wherein the oligonucleotide probe specific for the Swine Influenza virus comprises at least sixteen contiguous nucleotides of the sequence shown in SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or its complementary reverse sequences or a sequence having at least 70% sequence identity thereto.
45. The diagnostic kit, method for detecting or method of differentiating of any one of clauses 2, 5, 6 and 9 to 44, wherein the oligonucleotide probe specific for the Swine Influenza virus comprises at least eighteen contiguous nucleotides of the sequence shown in SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or a sequence having at least 70% sequence identity thereto.
46. The diagnostic kit, method for detecting or method of differentiating of any one of clauses 2, 5, 6 and 9 to 45, wherein the oligonucleotide probe specific for the Swine Influenza virus comprises at least twenty contiguous nucleotides of the sequence shown in SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or a sequence having at least 70% sequence identity thereto.
47. The diagnostic kit, method for detecting or method of differentiating of any one of clauses 2, 5, 6 and 9 to 46, wherein the oligonucleotide probe specific for the Swine Influenza virus comprises at least twenty-two contiguous nucleotides of the sequence shown in SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or a sequence having at least 70% sequence identity thereto.
48. The diagnostic kit, method for detecting or method of differentiating of any one of clauses 2, 5, 6 and 9 to 47, wherein the oligonucleotide probe specific for the Swine Influenza virus comprises at least twenty-four contiguous nucleotides of the sequence shown in SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or a sequence having at least 70% sequence identity thereto.
49. The diagnostic kit, method for detecting or method of differentiating of any one of clauses 2, 5, 6 and 9 to 48, wherein the oligonucleotide probe specific for the Swine Influenza virus comprises at least twenty-six contiguous nucleotides of the sequence shown in SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or a sequence having at least 70% sequence identity thereto.
50. The diagnostic kit, method for detecting or method of differentiating of any one of clauses 2, 5, 6 and 9 to 49, wherein the oligonucleotide probe specific for the Swine Influenza virus comprises at least twenty-eight contiguous nucleotides of the sequence shown in SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or a sequence having at least 70% sequence identity thereto.
51. The diagnostic kit, method for detecting or method of differentiating of any one of clauses 2, 5, 6 and 9 to 50, wherein the oligonucleotide probe specific for the Swine Influenza virus comprises a sequence shown in SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26) or a sequence having at least 70% sequence identity thereto.
52, The diagnostic kit, method for detecting or method of differentiating of any one of clauses 42 to 51, wherein said sequence identity of the oligonucleotide probe is at least 80%.
53. The diagnostic kit, method for detecting or method of differentiating of any one of clauses 42 to 52, wherein said sequence identity of the oligonucleotide probe is at least 90%.
54, The diagnostic kit, method for detecting or method of differentiating of any one of clauses 42 to 53, wherein said sequence identity of the oligonucleotide probe is at least 95%.
55. The diagnostic kit, method for detecting or method of differentiating of any one of clauses 42 to 54, wherein said sequence identity of the oligonucleotide probe is at least 97.5%.
56, The diagnostic kit, method for detecting or method of differentiating of any one of clauses 38 to 55, wherein the sequence of the oligonucleotide probe specific for the Swine Influenza virus comprises the sequence shown in SEQ ID NO:11; SEQ ID NO:13; SEQ ID NO:15; SEQ ID NO:17; SEQ ID NO:19; SEQ ID NO:21; SEQ ID NO:23 or SEQ ID NO:25 or its complementary reverse sequences (SEQ ID NO:12; SEQ ID NO:14; SEQ ID NO:16; SEQ ID NO:18; SEQ ID NO:20; SEQ ID NO:22; SEQ ID NO:24; SEQ ID NO:26).
57. The method for detecting or method of differentiating of any one of clauses 3 to 12 and 14 to 56, wherein the signal is an enzymatic signal, a fluorescent signal or an electrochemical signal.
58. The diagnostic kit, method for detecting or method of differentiating of any one of clauses 1 to 57, wherein the oligonucleotide probe or primer is coupled with a detectable label selected from the group consisting of a radioactive element and a fluorescent chemical.
59. The diagnostic kit, method for detecting or method of differentiating of any one of clauses 1 to 57, wherein the oligonucleotide probe is coupled with a detectable label selected from the group consisting of a radioactive element and a fluorescent chemical.
60. The diagnostic kit, method for detecting or method of differentiating of clause 58 or 59, wherein the fluorescent chemical label is selected from a fluorescein, a cyanine dye, a coumarin, a phycoerythrin, a phycobiliprotein, a dansyl chloride, a lanthanide complex or a fluorochrome.
61. The diagnostic kit, method for detecting or method of differentiating of clause 60, wherein said fluorochrome is R-phycoerythrin, Cy3, Cy5, Quasar 670, Rhodamin, Alexa, or Texas Red.
62. The diagnostic kit, method for detecting or method of differentiating of clause 60, wherein said fluorescein is 6-FAM (6-carboxyfluorescein), TET (6-carboxy-4,7,2′,7′-tetrachlorofluorescein), JOE (2,7-dimethoxy-4,5-dichloro-6-carboxyfluorescein) or HEX (6-carboxy-2′,4′,7′,4,7-hexachlorofluorescein).
63. The diagnostic kit, method for detecting or method of differentiating of any one of clauses 1 to 62, wherein the oligonucleotide probe is further labeled with a quencher selected from 6-carboxytetramethylrhodamine (TAMRA), black hole quencher (BHQ) BHQ-1 and 2 or 6-carboxy-X-rhodamine (ROX).
64. The diagnostic kit, method for detecting or method of differentiating of any one of clauses 1 to 63, wherein the oligonucleotide probe or primer is coupled with a fluorescent label.
65. The diagnostic kit, method for detecting or method of differentiating of any one of clauses 1 to 64, wherein the oligonucleotide probe or primer is coupled with a first coupling group.
66. The diagnostic kit, method for detecting or method of differentiating of any one of clauses 3 to 64, wherein the primer is coupled with a first coupling group.
67. The diagnostic kit, method for detecting or method of differentiating of clause 65 or 66, wherein the generation of a signal comprises providing a second coupling group.
68. The diagnostic kit, method for detecting or method of differentiating of any one of clauses 65 to 67, wherein said first and second coupling groups are selected from the group consisting of antibody-antigen, receptor-ligand, biotin-streptavidin, sugar-lectins, and complementary oligonucleotides.
69. The diagnostic kit, method for detecting or method of differentiating of any one of clauses 65 to 68, wherein the second coupling group is labelled.
70. The diagnostic kit, method for detecting or method of differentiating of clause 69, wherein the label is selected from the group consisting of a radioactive element, a fluorescent chemical or an enzyme.
71. The diagnostic kit, method for detecting or method of differentiating of clause 70, wherein said fluorescent chemical label is a fluorescent according to clauses 60 to 62.
72. The diagnostic kit, method for detecting or method of differentiating of clause 70, wherein said enzyme label is selected from horseradish peroxidase (HRP), esterase, alkaline phosphatase (AP), Glucose oxidase, β-galactosidase or Luciferase.
73, The diagnostic kit, method for detecting or method of differentiating of any one of clauses 3 to 12, 14 to 57, 65 to 70 and 72, wherein the oligonucleotide probe or primer signal is an enzymatic signal.
74. The diagnostic kit, method for detecting or method of differentiating of clause 70 or 72, wherein said enzyme converts a substrate into a reversible redox couple.
75. The diagnostic kit, method for detecting or method of differentiating of any one of clauses 3 to 12, 14 to 57, 65 to 70 and 72 to 74, wherein the oligonucleotide probe or primer signal is an electrochemical signal.
76. The diagnostic kit, method for detecting or method of differentiating of any one of clauses 3 to 12 and 14 to 75, wherein said amplification of polynucleotides is PCR (polymerase chain reaction) or real time PCR (polymerase chain reaction).
77. The diagnostic kit, method for detecting or method of differentiating of any one of clauses 3 to 76, wherein said forward and said reverse-oligonucleotide primer is specific for the NS (non-structural protein) gene segment.
78. The diagnostic kit, method for detecting or method of differentiating of any one of clauses 3 to 77, wherein said forward-oligonucleotide primer is specific for the NS-1 (non-structural protein-1) ORF.
79. The diagnostic kit, method for detecting or method of differentiating of any one of clauses 3 to 78, wherein said reverse-oligonucleotide primer is specific for the NS-2 (non-structural protein-2) ORF.
80. The diagnostic kit, method for detecting or method of differentiating of any one of clauses 3 to 79, wherein said forward and said reverse-oligonucleotide primer specific for the NS (non-structural protein) gene segment comprise at least twelve contiguous nucleotides of the sequence shown in SEQ ID NO:1 (gataataggctctctttgtg) or SEQ ID NO:2 (aggtaatggtgaaatttctc) or SEQ ID NO:27 to SEQ ID NO:38 or a sequence having at least 70% sequence identity thereto.
81. The diagnostic kit, method for detecting or method of differentiating of any one of clauses 3 to 80, wherein said forward and said reverse-oligonucleotide primer specific for the NS (non-structural protein) gene segment comprise at least fourteen contiguous nucleotides of the sequence shown in SEQ ID NO:1; SEQ ID NO:2 or SEQ ID NO:27 to SEQ ID NO:38 or a sequence having at least 70% sequence identity thereto.
82. The diagnostic kit, method for detecting or method of differentiating of any one of clauses 3 to 81, wherein said forward and said reverse-oligonucleotide primer specific for the NS (non-structural protein) gene segment comprise at least sixteen contiguous nucleotides of the sequence shown in SEQ ID NO:1; SEQ ID NO:2 or SEQ ID NO:27 to SEQ ID NO:38 or a sequence having at least 70% sequence identity thereto.
83. The diagnostic kit, method for detecting or method of differentiating of any one of clauses 3 to 82, wherein said forward and said reverse-oligonucleotide primer specific for the NS (non-structural protein) gene segment comprise at least eighteen contiguous nucleotides of the sequence shown in SEQ ID NO:1; SEQ ID NO:2 or SEQ ID NO:27 to SEQ ID NO:38 or a sequence having at least 70% sequence identity thereto.
84. The diagnostic kit, method for detecting or method of differentiating of any one of clauses 3 to 83, wherein said forward and said reverse-oligonucleotide primer specific for the NS (non-structural protein) gene segment comprise the sequence shown in SEQ ID NO:1; SEQ ID NO:2 or SEQ ID NO:27 to SEQ ID NO:38 or a sequence having at least 70% sequence identity thereto.
85. The diagnostic kit, method for detecting or method of differentiating of any one of clauses 80 to 84, wherein said sequence identity of the oligonucleotide primer is at least 80%.
86. The diagnostic kit, method for detecting or method of differentiating of any one of clauses 80 to 85, wherein said sequence identity of the oligonucleotide primer is at least 90%.
87. The diagnostic kit, method for detecting or method of differentiating of any one of clauses 80 to 86, wherein said sequence identity of the oligonucleotide primer is at least 95%.
88. The diagnostic kit, method for detecting or method of differentiating of any one of clauses 80 to 87, wherein said sequence identity of the oligonucleotide primer is at least 97.5%.
89, The diagnostic kit, method for detecting or method of differentiating of any one of clauses 80 to 88, wherein said forward and said reverse-oligonucleotide primer specific for the NS (non-structural protein) gene segment comprise the sequence shown in SEQ ID NO:1; SEQ ID NO:2 or SEQ ID NO:27 to SEQ ID NO:38.
90. The diagnostic kit, method for detecting or method of differentiating of any one of clauses 1 to 89, wherein said animal is swine.
91. The diagnostic kit, method for detecting or method of differentiating of any one of clauses 3 to 6, 11 to 12 and 14 to 90, wherein the biological sample is a nasal sample, oral fluid sample, respiratory tissue sample or lung sample.
   92. The diagnostic kit, method for detecting or method of differentiating of any one of clauses 7 to 12 and 14 to 90, wherein the environmental Sample is an air filter sample or a sample of a rope for collecting oral fluid.
93. The diagnostic kit, method for detecting or method of differentiating of any one of clauses 3 to 12 and 14 to 92, wherein the concentration of the modified live Swine Influenza virus specific vaccine or the Swine Influenza virus is between 2 to 12 log EID50.
94. The diagnostic kit, method for detecting or method of differentiating of any one of clauses 3 to 12 and 14 to 93, wherein the concentration of the modified live Swine Influenza virus specific vaccine or the Swine Influenza virus is between 4 to 10 log EID50.
95. The diagnostic kit, method for detecting or method of differentiating of any one of clauses 3 to 12 and 14 to 94, wherein the concentration of the modified live Swine Influenza virus specific vaccine or the Swine Influenza virus is between 6 to 8 log EID50.
95. The diagnostic kit, method for detecting or method of differentiating of any one of clauses 1 to 95, wherein the modified live Swine Influenza virus specific vaccine comprises a sequence which is identical or complementary to the oligonucleotide probe specific for the modified live Swine Influenza virus specific vaccine according to any one of clauses 14 to 34.
97. The diagnostic kit, method for detecting or method of differentiating of any one of clauses 1 to 96, wherein said identical or complementary sequence according to clause 96 is a non-naturally occurring sequence within the modified live Swine Influenza virus specific vaccine.
98. The diagnostic kit, method for detecting or method of differentiating of any one of clauses 1 to 97, wherein said identical or complementary sequence according to clause 96 or 97 is within the NS (non-structural protein) gene segment of the modified live Swine Influenza virus specific vaccine.
99. The diagnostic kit, method for detecting or method of differentiating of any one of clauses 1 to 98, wherein said identical or complementary sequence according to clause 96 to 98 is between the NS-1 (non-structural protein) and NS-2 ORF of the modified live Swine Influenza virus specific vaccine.
100. The diagnostic kit, method for detecting or method of differentiating of any one of clauses 1 to 99, wherein the modified live Swine Influenza virus specific vaccine is attenuated.
101. The diagnostic kit, method for detecting or method of differentiating of any one of clauses 1 to 100, wherein the modified live Swine Influenza virus specific vaccine is bivalent.
102. The diagnostic kit, method for detecting or method of differentiating of any one of clauses 1 to 101, wherein the modified live Swine Influenza virus specific vaccine comprises modified live H3N2 and H1N1 Swine Influenza virus.
103. The diagnostic kit, method for detecting or method of differentiating of any one of clauses 1 to 102, wherein the modified live H3N2 and H1N1 viruses of swine influenza virus have a deletion within the NS1 gene.
104. The diagnostic kit, method for detecting or method of differentiating of any one of clauses 1 to 103, wherein the modified live H3N2 and H1N1 viruses of swine influenza virus encode for a carboxy-terminal truncated NS1 protein.
105. The diagnostic kit, method for detecting or method of differentiating of any one of clauses 1 to 104, wherein the modified live H3N2 and H1N1 viruses of swine influenza virus encode for a carboxy-terminal truncated NS1 protein comprising NS1 amino acids 1 through 124, 1 through 125, 1 through 126, 1 through 127 or 1 through 128, wherein the amino terminal amino acid is number 1.
106 The diagnostic kit, method for detecting or method of differentiating of any one of clauses 1 to 105, wherein the modified live H3N2 and H1N1 viruses of swine influenza virus encode for a carboxy-terminal truncated NS1 protein comprising NS1 amino acids 1 through 126, wherein the amino terminal amino acid is number 1.
107. The diagnostic kit, method for detecting or method of differentiating of any one of clauses 1 to 106, wherein the modified live H3N2 and H1N1 viruses of swine influenza virus have a carboxy-terminal truncated NS1 protein resulting in a deletion of 91, 92, 93 or 94 amino acid residues from the carboxy terminus of NS1.
108. The diagnostic kit, method for detecting or method of differentiating of any one of clauses 1 to 107, wherein the modified live H3N2 and H1N1 viruses of swine influenza virus have a NS1 gene or protein from A/Swine/Texas/4199-2/98.
109. The diagnostic kit, method for detecting or method of differentiating of any one of clauses 1 to 108, wherein the modified live H3N2 virus of swine influenza is TX/98/del 126 containing the HA, NA, PB2, PB1, PA, NP, and M from A/Swine/Texas/4199-2/98 and the NS1-126 gene is from A/Swine/Texas/4199-2/98 and, wherein the modified live H1N1 virus of swine influenza contains HA and NA from A/swine/Minnesota/37866/1999 (H1N1) and PB2, PB1, PA, NP, M from A/Swine/Texas/4199-2/98 (H3N2) and the NS1-126 gene is from A/Swine/Texas/4199-2/98 (H3N2).
110. The diagnostic kit of any one of clauses 13 to 109, wherein said at least one forward and one reverse-oligonucleotide primer pair and said oligonucleotide probe specific for the modified live Swine Influenza virus specific vaccine are in one container.
111. The diagnostic kit of any one of clauses 13 to 109, wherein said at least one forward and one reverse-oligonucleotide primer pair and said oligonucleotide probe specific for the modified live Swine Influenza virus specific vaccine are in two or more separate containers.
112. The diagnostic kit of any one of clauses 13 to 109, wherein said at least one forward and one reverse-oligonucleotide primer pair, said oligonucleotide probe specific for the modified live Swine Influenza virus specific vaccine and said oligonucleotide probe specific for the Swine Influenza virus are in one container.
113. The diagnostic kit of any one of clauses 13 to 109, wherein said at least one forward and one reverse-oligonucleotide primer pair, said oligonucleotide probe specific for the modified live Swine Influenza virus specific vaccine and said oligonucleotide probe specific for the Swine Influenza virus are in two or more separate containers.
114. The diagnostic kit of any one of clauses 2 and 13 to 109, wherein said oligonucleotide probe specific for the modified live Swine Influenza virus specific vaccine and said oligonucleotide probe specific for the Swine Influenza virus are in one container.
115. The diagnostic kit of any one of clauses 2 and 13 to 109, wherein said oligonucleotide probe specific for the modified live Swine Influenza virus specific vaccine and said oligonucleotide probe specific for the Swine Influenza virus are in two or more separate containers.
116. The diagnostic kit of any one of clauses 1 or 2 and 13 to 115, wherein said kit comprises one or more control samples.
117. The diagnostic kit of clause 116, wherein said control sample is a RNA, cDNA or DNA sample.
118. The diagnostic kit of clause 116 or 117, wherein the control is a positive control comprising RNA, cDNA or DNA specific for the modified live Swine Influenza virus specific vaccine.
119, The diagnostic kit of clause 116 or 117, wherein the control is a positive control comprising RNA, cDNA or DNA specific for the Swine Influenza virus.
120. The diagnostic kit of any one of clauses 1 or 2 and 13 to 119, wherein said kit comprises an instruction letter providing information for use of the kit.
121. The diagnostic kit, method for detecting or method of differentiating of any one of clauses 1 to 118, wherein said Swine Influenza virus is a Swine Influenza A virus.

### EXAMPLES

The following examples are only intended to illustrate the present invention. They shall not limit the scope of the claims in any way.

### Materials and Methods

1. Preparation of the Primers/Probes Mix

TABLE 1
Primers/Probes sequences:

| | |
|---|---|
| NSfor | 5′-gataataggctctctttgtg-3′ |
| | (SEQ ID NO: 1) |
| NSrev | 5′-aggtaatggtgaaatttctc-3′ |
| | (SEQ ID NO: 2) |
| MLV probe 1 | Quasar 670 5′-atggaaaagtagatcttgatta |
| | attaagagg-3′ BHQ2 |
| | (SEQ ID NO: 7) |
| MLVfluprobe2 | Quasar 670 5′-agtagatcttgattaattaaga |
| | gggagc-3′ BHQ-2 |
| | (SEQ ID NO: 5) |
| WTfluprobe | Fam 5′-gtgtgatctttaaccgattagagacttt |
| | g-3′ BHQ-1 |
| | (SEQ ID NO: 11) |

TABLE 2
Primers/Probes concentration

| Primers/Probes | | Final |
|---|---|---|
| Mix | | concentration: |
| NSfor | gataataggctctctttgtg (SEQ ID NO: 1) | 0.5 μM |
| NSrev | aggtaatggtgaaatttctc (SEQ ID NO: 2) | 0.4 μM |
| WTfluprobe | gtgtgatctttaaccgattagagactttg (SEQ ID NO: 11) | 0.25 μM |
| MLVfluprobe1 | atggaaaagtagatcttgattaattaagagg (SEQ ID NO: 7) | 0.25 μM |
| MLVfluprobe2 | agtagatcttgattaattaagagggagc (SEQ ID NO: 5) | 0.25 μM |

Primers and probes were purchased from Biosearch Technologies.
NS (non-structural protein); for (forward); rev (reverse); WT (wildtype); MLV (modified live virus)

2. Preparation of the Master Mix

TABLE 3
Preparation of the master mix

| Life Tech FAST viral mix | 1x (μl) |
|---|---|
| Fast viral Mix | 3 |
| Multiplex RT-PCR Enzyme Mix | 0.5 |
| Primer mix | 1.0 |
| Probe or probe mix (WT and/or MLV) | 1.0 |
| Template RNA | 5.5 |
| Final volume | 11.0 |

5.5 μl mix + 5.5 μl RNA

3. Cycling Protocol

TABLE 4
Cycling protocol

| | | | Sample Ramp | | |
|---|---|---|---|---|---|
| Step | Time | Temperature | Rate | | |
| RT | 5′ | 50° C. | 4.4 | | |
| Initial Denaturation | 20″ | 95° C. | 4.4 | | |
| Denaturation | 3″ | 95° C. | 4.4 | [symbol] | 40x |
| Annealing/Extension | 45″ | 60° C. | 2.2 | | |
| Cooling | 20″ | 40° C. | 4.4 | | |

4. Extraction Information

Samples were extracted using the Life Tech CORE kit (Thermofisher Scientific).

TABLE 5
Extraction Oral Fluids

| Step | Core Oral Fluids Procedure |
|---|---|
| 1 | Add 450 μL of Lysis solution to 96 deep well plate |
| 2 | Load 300 μL sample |
| 3 | Shake 3 minutes, spin 3 minutes |
| 4 | In a new 96 dw plate, load 30 μL of beads |
| 5 | Add 600 μL of clarified lysate from step 3 to beads |
| 6 | Add 350 μL of ISO to samples |
| 7 | Load on KF |

TABLE 6
Extraction Nasal Swab

| Step | Core (Serum) Swab Procedure |
|---|---|
| 1 | Load 30 μL bead mix into deep well plate |
| 2 | Load 100 μl of sample to beads |
| 3 | Add 700 μL lysis/binding solution to samples |
| 4 | Load on KF |

5. Equipment Used

Samples were extracted using the KingFisher FLEX 96 robot (Thermofisher Scientific).
Real-Time PCR was conducted using a Lightcycler 480 system 2 (Roche Applied Science)

6. Principle of Detection

Two hydrolysis probes are designed to bind downstream of the primers during the PCR reaction. The 5′ end of each probe is labeled with a fluorescent reporter molecule (see Table 1). On the 3′ end, the probe has been labeled with a quencher that limits the fluorescent output. During the PCR reaction, the reporter and quencher are cleaved by the polymerase enzyme.
The WT probe is labeled with a FAM reporter which has a peak excitation at a wave length of 495 nanometers (nm) and a peak emission of 520 nm. The MLV probe is labeled with a Quasar 670 reporter which has a peak excitation at a wave length of 647 nm and a peak emission of 670 nm. These reporter dyes and quenchers are recommended for use on the Lightcycler 480 II (Roche), but other commonly used dyes may also be used.

TABLE 7
Information on samples tested

| Virus Information | Concentration | Code |
|---|---|---|
| Provenza vaccine | 6-8 log EID50 | A |
| A/Swine/Indiana/1726/1988 (H1N1) | 6-8 log EID50 | B |
| A/Swine/Texas/4199-2/1998 (H3N2) | 6-8 log EID50 | C |
| A/Swine/Nebraska/97901-10/2008 (H3N2) | 6-8 log EID50 | D |
| A/Swine/North Carolina/001169/2006 (H1N2) | 6-8 log EID50 | E |

Whole virus sequencing for all viruses mentioned in table 7 were done at Newport Labs to confirm probe match with WT probe
The Provenza vaccine is a bivalent SIAV vaccine that already has been described in WO 2016/137929 A1.

### Study Design—Experiment #1

A spike study was created where each of the above viruses was spiked into negative nasal swab media or negative oral fluid samples. A 1:10 dilution series for each spiked sample was created with in the appropriate sample. Exemplary, A is the undiluted (Provenza vaccine) sample, A1 is the 1:10 dilution, A2 is the 1:100 dilution and so forth. Each sample was extracted one time and tested by qPCR (quantitative PCR) in triplicate using the master mix (WT and Provenza probe) and cycling protocol as described above. The average Ct (cycle threshold) value of the 3 replicates was reported.

### Study Design—Experiment #2

Samples from Experiment #1 were mixed and tested to assess cross reactivity and detection to mimic wild infection of influenza virus around the time of vaccination. Samples were tested by qPCR in triplicate and the average Cycle threshold (Ct) values were reported.

### Study Design—Experiment #3

Experiment #3 was conducted to assess the potential use of an alternative Probe design called MLV probe 1 (atggaaaagtagatcttgattaattaagagg). A 1:10 dilution series (provenza 1 to provenza 5) was created using the Ingelvac Provenza vaccine. PCR was performed which compared results of the MLV1 and MLV2 probe designs. Samples were tested in duplicate and the average cycle threshold (Ct) was reported for comparison.

### Experiment #1 Results—Nasal Swab Samples:

TABLE 8
Provenza (MLV)

| | WT | MLV |
|---|---|---|
| A | Not detected | 22.75 |
| A1 | Not detected | 22.43 |
| A2 | Not detected | 26.14 |
| A3 | Not detected | 26.56 |
| A4 | Not detected | 31.03 |

TABLE 9
H1N1 Ind 88 (WT)

| | WT | MLV |
|---|---|---|
| B | 30.56 | Not detected |
| B1 | 31.73 | Not detected |
| B2 | 35.65 | Not detected |
| B3 | 38.13 | Not detected |
| B4 | 41.02 | Not detected |

TABLE 10
H3N2 Tx 98 (WT)

| | WT | MLV |
|---|---|---|
| C | 23.01 | Not detected |
| C1 | 25.20 | Not detected |
| C2 | 29.08 | Not detected |
| C3 | 32.35 | Not detected |
| C4 | 35.34 | Not detected |

TABLE 11
H3N2 NE 08 (WT)

| | WT | MLV |
|---|---|---|
| D | 18.59 | Not detected |
| D1 | 23.31 | Not detected |
| D2 | 28.11 | Not detected |
| D3 | 32.10 | Not detected |
| D4 | 35.94 | Not detected |

TABLE 12
H1N2 NC 06 (WT)

| | WT | MLV |
|---|---|---|
| E | 20.51 | Not detected |
| E1 | 22.09 | Not detected |
| E2 | 29.58 | Not detected |
| E3 | 33.45 | Not detected |
| E4 | 35.92 | Not detected |

### Experiment #1 Results—Oral Fluid Samples:

TABLE 13
Provenza (MLV)

| | WT | MLV |
|---|---|---|
| A | Not detected | 20.42 |
| A1 | Not detected | 25.86 |
| A2 | Not detected | 26.80 |
| A3 | Not detected | 30.87 |
| A4 | Not detected | 35.12 |

TABLE 14
H1N1 Ind 88 (WT)

| | WT | MLV |
|---|---|---|
| B | 23.68 | Not detected |
| B1 | 29.75 | Not detected |
| B2 | 32.61 | Not detected |
| B3 | 36.01 | Not detected |
| B4 | 39.68 | Not detected |

TABLE 15
H3N2 Tx 98 (WT)

| | WT | MLV |
|---|---|---|
| C | 17.90 | Not detected |
| C1 | 21.68 | Not detected |
| C2 | 25.87 | Not detected |
| C3 | 29.16 | Not detected |
| C4 | 33.24 | Not detected |

TABLE 16
H3N2 NE 08 (WT)

| | WT | MLV |
|---|---|---|
| D | 14.91 | Not detected |
| D1 | 20.45 | Not detected |
| D2 | 25.50 | Not detected |
| D3 | 29.24 | Not detected |
| D4 | 32.43 | Not detected |

TABLE 17
H1N2 NC 06 (WT)

| | WT | MLV |
|---|---|---|
| E | 16.10 | Not detected |
| E1 | 21.06 | Not detected |
| E2 | 28.32 | Not detected |
| E3 | 31.51 | Not detected |
| E4 | 35.17 | Not detected |

Experiment #2 Results—Provenza Strong to Weak Mixed with WT Virus Strong to Weak:

TABLE 18
Provenza (MLV) + H1N1 Ind 88 (WT)

| | |
|---|---|
| Nasal Swab | Oral Fluid |

| | WT | MLV | WT | MLV |
|---|---|---|---|---|
| A + B | 33.81 | 26.73 | 29.57 | 25.86 |
| A1 + B1 | 38.64 | 30.44 | 34.76 | 31.98 |
| A2 + B2 | 33.36 | 33.40 | 33.77 | 32.91 |
| A3 + B3 | 40.43 | 36.92 | Not detected | 37.60 |
| A4 + B4 | 41.66 | 38.23 | Not detected | 37.90 |

TABLE 19
Provenza (MLV) + H3N2 Tx 98 (WT)

| | |
|---|---|
| Nasal Swab | Oral Fluid |

| | WT | MLV | WT | MLV |
|---|---|---|---|---|
| A + C | 27.65 | 30.49 | 21.47 | 28.97 |
| A1 + C1 | 29.33 | 34.70 | 26.89 | 35.49 |
| A2 + C2 | 29.99 | 36.26 | 27.21 | 36.47 |
| A3 + C3 | 34.47 | 39.89 | 32.18 | 40.32 |
| A4 + C4 | 38.01 | 42.09 | 36.32 | 42.89 |

### Experiment #3 Results—MLV Probe Comparison:

TABLE 20

| WT probe | | | Provenza Probe | | |
|---|---|---|---|---|---|
| | Ct | Ct | | Ct | Ct |
| Name | MLV1 | MLV2 | Name | MLV1 | MLV2 |
| WT Pos Ctrl | 34.67 | 34.65 | WT Pos Ctrl | | |
| Neg Ctrl | | | Neg Ctrl | | |
| Provenza | | | Provenza | 21.88 | 21.48 |
| undiluted | | | undiluted | | |
| provenza -1 | | | provenza -1 | 25.75 | 24.84 |
| provenza -2 | | | provenza -2 | 29.95 | 28.74 |
| provenza -3 | | | provenza -3 | 32.82 | 31.9 |
| provenza -4 | | | provenza -4 | 37.99 | 33.93 |
| provenza -5 | | | provenza -5 | 40.79 | 37.75 |
| WT Pos Ctrl | 36.07 | 35.53 | Pos Ctrl | | |
| Neg Ctrl | | | Neg Ctrl | | |
| Provenza | | | Provenza | 20.1 | 21.84 |
| undiluted | | | undiluted | | |
| provenza -1 | | | provenza -1 | 26.08 | 25.58 |
| provenza -2 | | | provenza -2 | 29.82 | 28.79 |
| provenza -3 | | | provenza -3 | 32.8 | 32.45 |
| provenza -4 | | | provenza -4 | 34.2 | 35.89 |
| provenza -5 | | | provenza -5 | | |

### Discussion and Conclusions

The results for experiment #1 show that when only Provenza (MLV) is present in a sample, it is the only virus detected with the MLV probe. Further, when a wild strain of influenza virus is the only virus present it is only detected with the WT probe. Thus, the probes are specific for detection of WT virus and MLV respectively.
Further, this experiment demonstrates that WT and MLV virus can be detected in different samples such as nasal swab samples and oral fluid samples, but could also be detected in other samples such as respiratory tissues, or environmental samples.
Furthermore, the virus can be detected in multiple dilutions of a sample.
Thus, Experiment #1 shows that the assay can detect the correct virus using the intended probe in different samples at various dilutions. There is no interference between the different probes.
The conclusion for Experiment #2 is that the assay can detect and differentiate influenza viruses (Ingelvac Provenza vaccine from wild type influenza A viruses) in different samples at various dilutions. There is no interference between the different probes.
The conclusion for experiment 3 is that an alternate probe design for the detection of the Ingelvac Provenza vaccine is possible. The results from table 20 show a slightly improved detection using MLV2. Neither probe design cross reacts with the WT probe to produce unwanted signal in that detection channel.

### Experiment #4—Field Study Summary

Study design—Experiment #4
Field validation—Wean age vaccination
A farm was identified where wean age (3 weeks of age) piglets were vaccinated with Provenza per label upon arrival at a finishing unit. 5 animals per each of the 9 pens in barn 1 and barn 2 were nasal swabbed on the following days post vaccination: 0, 1, 2, 3, 4, 5, 6, 7, 8, 10, 12, 14, 17, and 21. Prior to the first collection, animals were ear tagged so the same animals could be sampled over the course of the study. Additionally, 1 cotton rope was hung per pen on the above collection days to collect pen level oral fluids. All samples were tested by 2 PCR tests: IAV-S screen PCR from Life Technologies (Matrix and Nucleoprotein targets) according to manufacturer instruction as well as Provenza™ PCR (NS1 target) as described above.
Using nasal swabs, PCR positives can be detected to 4 days post-vaccination (data not shown). Using oral fluids, positives can be detected to 10 days post-vaccination. The data suggests that both oral fluids and nasal swabs can be used for testing in the field to measure vaccination status of a herd in young piglets.

### Field Validation—Processing Age Vaccination

A customer farm was identified where young piglets (3-8 days of age, average is 4) were vaccinated with Provenza™ per label. 5 animals per farrowing crate were nasal swabbed on the following days post vaccination: 0, 1, 2, 3, 4, 5, 6, 7, 8, 10, 12, 14, 17, and 21. Prior to the first collection, animals were ear tagged so the same animals could be sampled over the course of the study. Sow ID numbers were also recorded. All samples were tested by 2 PCR tests: IAV-S screen PCR from Life Technologies (Matrix and Nucleoprotein targets) according to manufacturer instruction as well as Provenza™ PCR (NS1 target) as described above.
Using nasal swabs, PCR positives can be detected to 14 days post-vaccination (data not shown). The data suggests that testing in the field to measure vaccination status of a herd in weaned animals works as well.

### Experiment #5 Mobinostics Method

### Materials and Methods

1. Primers/Probes Sequences:

NSfor:
(SEQ ID NO: 37)
5′ gataataggctctctttgtgtgc 3′
NSrev:
(SEQ ID NO: 38)
5′Biotin-gagaaggtaatggtgaaatttctc 3′
CMOS Thiol Probes:
5′ ttttttttttttttttttttttttttttttttttttttttAGTAGAT
CTTGATTAATTAAGAGGGAGCAATCG 3′
(SEQ ID NO: 39: AGTAGATCTGATTAATTAAGAGGGAGCAATCG)

Primers and probes were purchased from Metabion.

2. Reagents, RT-PCR Cycles

All reagents (primers, probes, master mix) and cycling conditions are integrated into the Mobinostics card.
The two Provenza components were analyzed separately on RNA basis (H3N2 RNA component and H1N1 RNA component). First, the RNA copies in the RNA samples were determined by reference influenza virus RNAs based on NP RNA standard. A 1535nt long NP (Nucleoprotein) RNA standard was ordered from Eurofins. 1e08 NP RNA copy was prepared as a stock and aliquoted in the way that each aliquot was thawed one time. Serial dilutions (1e08-1e02 NP RNA copies) were prepared for the quantitative real time RT-PCR. The one-step real time RT-PCR was performed with NP primers and TaqMan NP probe using 4×TagMan Fast Virus 1-Step Master Mix. The RNA copies of reference influenza virus RNAs were calculated based on the NP standard curve.
Then, 20 RNA copies/μl, 200 RNA copies/μl, 1000 RNA copies/μl and 10000 RNA copies/μl were applied to the Mobinostics card and measured in 4-10 technical replicates on the Mobinostics device.
The CMOS Chip technology has been well described in the prior art. Exemplary, WO 2018/065104 A1, Roland Thewes (Enabling CMOS-based DNA array chips) and Frey et al 2005 (A Digital CMOS DNA Chip) describe the CMOS technology. In general, redox cycling techniques comprise chip technologies such as exemplary the CMOS Chip technology. In general, the electrochemical principle behind this is an enzyme-label-based, current-generation process, so that hybridization of complementary DNA strands translates into sensor currents at the sensor electrodes between 1 pA to 100 nA. Probe molecules are immobilized on the surface of a sensor element. The amplification product tagged by biotin label (by biotin labelled primers) is applied to the chip. After the hybridization and washing phases, Streptavidin-AP is applied to the chip. After incubation and washing steps, a chemical substrate (para-aminophenyl-phosphate) is applied to the chip. The enzyme label, available at the sites where hybridization occurred, cleaves the phosphate group and the electrochemically active para-aminophenol is generated. Simultaneously applying an oxidation and a reduction potential to the sensor electrodes, para-aminophenol is oxidized to quinoneimine at the one electrode, and quinoneimine is reduced to para-aminophenol at the other one.

TABLE 21
Mobinostics results of the H1N1 and H3N2 components:

| Sample: | Mean MLV-NS | Standard deviation |
|---|---|---|
| Negative control | −0.08 | 0.08 |
| H1N1 MLV 20 RNA copies/μl | 0.17 | 0.15 |
| H1N1 MLV 200 RNA copies/μl | 0.73 | 0.05 |
| H1N1 MLV 1000 RNA copies/μl | 0.78 | 0.04 |
| H1N1 MLV 10000 RNA copies/μl | 0.84 | 0.02 |
| H3N2 MLV 20 RNA copies/μl | −0.09 | 0.06 |
| H3N2 MLV 200 RNA copies/μl | 0.13 | 0.33 |
| H3N2 MLV 1000 RNA copies/μl | 0.52 | 0.09 |
| H3N2 MLV 10000 RNA copies/μl | 0.79 | 0.01 |

The results as shown in Table 21 show that also DNA Chip technology can be used for detecting RNA/DNA components of Provenza.

1. (canceled)

2. (canceled)

3. A method for detecting an animal vaccinated with a modified live Swine Influenza virus specific vaccine in a biological sample comprising the steps of:
a. obtaining a biological sample containing at least one nucleic acid from an animal;
b. providing at least one forward and a reverse-oligonucleotide primer pair and an oligonucleotide probe specific for the modified live Swine Influenza virus specific vaccine for detecting a vaccination with a Swine Influenza virus specific vaccine, said oligonucleotide probe comprises at least fourteen contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto;
c. contacting said oligonucleotide primer pair with said biological sample under conditions which allow for amplification of polynucleotides;
d. generating a signal using said oligonucleotide probe specific for the modified live Swine Influenza virus specific vaccine; and
e. detecting said signal, wherein detection of said signal indicates a vaccination with a Swine Influenza virus specific vaccine in the biological sample.

4. The method of claim 3 further providing an oligonucleotide probe specific for the Swine Influenza virus for detecting an infection with Swine Influenza virus; and
a. contacting said oligonucleotide primer pair with said biological sample under conditions which allow for amplification of polynucleotides;
b. generating a signal using said oligonucleotide probe specific for the modified live Swine Influenza specific vaccine and/or said oligonucleotide probe specific for the Swine Influenza virus; and
c. differentiating animals vaccinated with a modified live Swine Influenza virus specific vaccine from animals infected with Swine Influenza virus by detecting said signal, wherein
i) detection of a signal using said oligonucleotide probe specific for the modified live Swine Influenza specific vaccine indicates a vaccination with a Swine Influenza specific vaccine in the biological sample, and,
ii) detection of signal using said oligonucleotide probe specific for the Swine Influenza virus indicates an infection with a Swine Influenza virus in the biological sample.

5. The method of claim 3 further comprising detecting animals vaccinated within a group of animals comprising the steps of:
a. obtaining an environmental Sample containing at least one nucleic acid from an animal;
b. contacting said oligonucleotide primer pair and said oligonucleotide probe with said environmental Sample under conditions which allow for amplification of polynucleotides;
c. generating a signal using said oligonucleotide probe specific for the modified live Swine Influenza specific vaccine; and
d. detecting the oligonucleotide probe signal, wherein the presence of the oligonucleotide probe signal indicates a vaccination with the Swine Influenza virus specific vaccine within said group of animals.

6. A method for determining a ratio between animals vaccinated with a modified live Swine Influenza virus specific vaccine and animals infected with Swine Influenza virus within a group of animals comprising the steps of:
a. obtaining an environmental Sample containing at least one nucleic acid from an animal;
b. providing
i) at least one forward and one reverse-oligonucleotide primer pair, and,
ii) an oligonucleotide probe specific for the modified live Swine Influenza virus specific vaccine for detecting a vaccination with a Swine Influenza virus specific vaccine, said oligonucleotide probe comprises at least fourteen contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto, and,
iii) an oligonucleotide probe specific for the Swine Influenza virus for detecting an infection with Swine Influenza virus;
c. contacting said oligonucleotide primer pair and said oligonucleotide probes with said environmental Sample under conditions which allow for amplification of polynucleotides;
d. generating a signal using said oligonucleotide probe specific for the modified live Swine Influenza specific vaccine and/or said oligonucleotide probe specific for the Swine Influenza virus; and
e. detecting the oligonucleotide probe signal from
i) the oligonucleotide probe specific for the modified live Swine Influenza virus specific vaccine, and,
ii) from the oligonucleotide probe specific for the Swine Influenza virus;
f. generating a ratio of i) and ii) or ii) and i) of step e.

7. (canceled)

8. (canceled)

9. (canceled)

10. The method for detecting of claim 3, wherein the oligonucleotide probe specific for the modified live Swine Influenza virus specific vaccine comprises at least twelve contiguous nucleotides of the sequence shown in SEQ ID NO:3 or its reverse complementary sequence (SEQ ID NO:4) or a sequence having at least 70% sequence identity thereto.

11. The method for detecting of claim 3, wherein the oligonucleotide probe specific for the modified live Swine Influenza virus specific vaccine binds to a non-naturally occurring sequence within the modified live Swine Influenza specific vaccine within the NS (non-structural protein) gene segment.

12. The method for detecting of claim 11, wherein the signal is an enzymatic signal, a fluorescent signal or an electrochemical signal.

13. The method for detecting of claim 12, wherein said amplification of polynucleotides is PCR (polymerase chain reaction) or real time PCR (polymerase chain reaction).

14. The method for detecting of claim 11, wherein said forward and said reverse-oligonucleotide primer is specific for the NS (non-structural protein) gene segment.

15. The method for detecting of claim 10, wherein said animal is swine.

16. The method for detecting of claim 10, wherein the biological sample is a nasal sample, oral fluid sample, respiratory tissue sample or lung sample.

17. The method for detecting of claim 5, wherein the environmental Sample is an air filter sample or a sample of a rope for collecting oral fluid.

18. The method for detecting of claim 17, wherein the oligonucleotide probe specific for the modified live Swine Influenza virus specific vaccine comprises at least twelve contiguous nucleotides of the sequence shown in SEQ ID NO:3 or its reverse complementary sequence (SEQ ID NO:4) or a sequence having at least 70% sequence identity thereto.

19. The method for detecting of claim 3, wherein the concentration of the modified live Swine Influenza virus specific vaccine or the Swine Influenza virus is between 2 to 12 log EID50.

20. The method for detecting of claim 19, wherein the oligonucleotide probe specific for the modified live Swine Influenza virus specific vaccine comprises at least twelve contiguous nucleotides of the sequence shown in SEQ ID NO:3 or its reverse complementary sequence (SEQ ID NO:4) or a sequence having at least 70% sequence identity thereto.

21. A diagnostic kit for the detection of an animal vaccinated with a modified live Swine Influenza virus specific vaccine comprising an oligonucleotide probe specific for the modified live Swine Influenza virus specific vaccine comprising at least fourteen contiguous nucleotides of the sequence shown in SEQ ID NO:3 (tagatcttgattaattaa) or its reverse complementary sequence (SEQ ID NO:4 ttaattaatcaagatcta) or a sequence having at least 70% sequence identity thereto.

22. The diagnostic kit of claim 20 further comprising an oligonucleotide probe specific for the Swine Influenza virus for detecting an infection with Swine Influenza virus, wherein said oligonucleotide probe is used for differentiating animals vaccinated with a modified live Swine Influenza virus specific vaccine from animals infected with Swine Influenza virus.

23. The diagnostic kit according to claim 21, wherein said kit comprises at least one forward and reverse-oligonucleotide primer pair.

24. The diagnostic kit of claim 22, wherein said kit comprises at least one forward and reverse-oligonucleotide primer pair, and wherein said at least one forward and one reverse-oligonucleotide primer pair, said oligonucleotide probe specific for the modified live Swine Influenza virus specific vaccine and said oligonucleotide probe specific for the Swine Influenza virus are in one container.

25. The diagnostic kit of claim 24, wherein said at least one forward and one reverse-oligonucleotide primer pair, said oligonucleotide probe specific for the modified live Swine Influenza virus specific vaccine and said oligonucleotide probe specific for the Swine Influenza virus are in two or more separate containers.


SEQUENCE LISTING (40 sequences)

SEQ ID NO: 1 - DNA, 20 bp, swine influenza virus
gataataggctctctttgtg

SEQ ID NO: 2 - DNA, 20 bp, swine influenza virus
aggtaatggtgaaatttctc

SEQ ID NO: 3 - DNA, 18 bp, Artificial Sequence
  Primer
tagatcttgattaattaa

SEQ ID NO: 4 - DNA, 18 bp, Artificial Sequence
  primer
ttaattaatcaagatcta

SEQ ID NO: 5 - DNA, 28 bp, Artificial Sequence
  primer
agtagatcttgattaattaagagggagc

SEQ ID NO: 6 - DNA, 28 bp, Artificial Sequence
  primer
gctccctcttaattaatcaagatctact

SEQ ID NO: 7 - DNA, 31 bp, Artificial Sequence
  primer
atggaaaagtagatcttgattaattaagagg

SEQ ID NO: 8 - DNA, 31 bp, Artificial Sequence
  primer
cctcttaattaatcaagatctacttttccat

SEQ ID NO: 9 - DNA, 33 bp, Artificial Sequence
  primer
agtagatcttgattaattaagagggagcaatcg

SEQ ID NO: 10 - DNA, 33 bp, Artificial Sequence
  primer
cgattgctccctcttaattaatcaagatctact

SEQ ID NO: 11 - DNA, 29 bp, swine influenza virus
gtgtgatctttaaccgattagagactttg

SEQ ID NO: 12 - DNA, 29 bp, swine influenza virus
caaagtctctaatcggttaaagatcacac

SEQ ID NO: 13 - DNA, 25 bp, swine influenza virus
tgatactactaagggctttcactga

SEQ ID NO: 14 - DNA, 25 bp, swine influenza virus
tcagtgaaagcccttagtagtatca

SEQ ID NO: 15 - DNA, 25 bp, swine influenza virus
tgatactactaagagctttcactga

SEQ ID NO: 16 - DNA, 25 bp, swine influenza virus
tcagtgaaagctcttagtagtatca

SEQ ID NO: 17 - DNA, 25 bp, swine influenza virus
taatactactaagggctttcactga

SEQ ID NO: 18 - DNA, 25 bp, swine influenza virus
tcagtgaaagcccttagtagtatta

SEQ ID NO: 19 - DNA, 25 bp, swine influenza virus
tgatactactgagagctttcactga

SEQ ID NO: 20 - DNA, 25 bp, swine influenza virus
tcagtgaaagctctcagtagtatca

SEQ ID NO: 21 - DNA, 24 bp, swine influenza virus
tggtactactaagggctttcactg

SEQ ID NO: 22 - DNA, 24 bp, swine influenza virus
cagtgaaagcccttagtagtacca

SEQ ID NO: 23 - DNA, 24 bp, swine influenza virus
tgatactactaagggctttcaccg

SEQ ID NO: 24 - DNA, 24 bp, swine influenza virus
cggtgaaagcccttagtagtatca

SEQ ID NO: 25 - DNA, 24 bp, swine influenza virus
tgatactactgagggctttcactg

SEQ ID NO: 26 - DNA, 24 bp, swine influenza virus
cagtgaaagccctcagtagtatca

SEQ ID NO: 27 - DNA, 20 bp, swine influenza virus
gataataggccctctttgtg

SEQ ID NO: 28 - DNA, 19 bp, swine influenza virus
gataataggccctctttgc

SEQ ID NO: 29 - DNA, 20 bp, swine influenza virus
gataacaggctctctttgtg

SEQ ID NO: 30 - DNA, 18 bp, swine influenza virus
caataggccctctttgtg

SEQ ID NO: 31 - DNA, 22 bp, swine influenza virus
gataataggctttctttgtgtg

SEQ ID NO: 32 - DNA, 20 bp, swine influenza virus
aggcaatggtgaaatttctc

SEQ ID NO: 33 - DNA, 22 bp, swine influenza virus
aaggtaatgatgaaatttctcc

SEQ ID NO: 34 - DNA, 20 bp, swine influenza virus
aggtaatggtgaaatttcac

SEQ ID NO: 35 - DNA, 20 bp, swine influenza virus
aggtaatggtgagatttctc

SEQ ID NO: 36 - DNA, 20 bp, swine influenza virus
aggtaagggtgaaatttctc

SEQ ID NO: 37 - DNA, 23 bp, Artificial Sequence
  primer
gataataggctctctttgtgtgc

SEQ ID NO: 38 - DNA, 24 bp, Artificial Sequence
  primer
gagaaggtaatggtgaaatttctc

SEQ ID NO: 39 - DNA, 33 bp, Artificial Sequence; identical to SEQ ID NO: 9
  primer

SEQ ID NO: 40 - DNA, 33 bp, Artificial Sequence; identical to SEQ ID NO: 10
  primer